(19) United States
(12) Patent Application Publication
(10) Pub. No.: US 2025/0269077 A1
(43) Pub. Date: Aug. 28, 2025

(54) BACTERIA OR VIRUSES INACTIVATION DEVICE, TREATMENT DEVICE, DISCHARGE LAMP

(71) Applicant: Ushio Denki Kabushiki Kaisha, Tokyo (JP)

(72) Inventors: Yoshihiko OKUMURA, Tokyo (JP); Hideaki YAGYU, Tokyo (JP); Kiyoyuki KABUKI, Tokyo (JP)

(73) Assignee: Ushio Denki Kabushiki Kaisha, Tokyo (JP)

(21) Appl. No.: 18/857,758

(22) PCT Filed: Apr. 17, 2023
(86) PCT No.: PCT/JP2023/015332

§ 371 Date: Oct. 17, 2024

(30) Foreign Application Priority Data
Apr. 18, 2022 (JP) 2022-068210
Sep. 8, 2022 (JP) 2022-142876

(51) Int. Cl. A61L 2/10; A61L 2/02

(52) U.S. Cl. CPC A61L 2/10; A61L 2/022

(57) ABSTRACT

Disclosed herein is a device capable of irradiating a narrower area than ever before with ultraviolet light to inactivate bacteria or viruses. This device includes a first light source that emits ultraviolet light having a principal wavelength band overlapping a range of 200 nm or more and less than 240 nm; a housing body that houses the first light source; and a light guide body that has an elongated shape and guides the ultraviolet light emitted from the light source in a longitudinal direction, part of the light guide body, including a first end that is an end on a side close to the light source, being positioned in the housing body. The light guide body is disposed in such a manner that the second end, which is an end on a side opposite to the first end, projects outside the housing body.

### TECHNICAL FIELD

The present invention relates to a bacteria or viruses inactivation device. The present invention also relates to a treatment device including the inactivation device. The present invention also relates to a discharge lamp.

### BACKGROUND ART

The present applicant has proposed a small ultraviolet irradiation apparatus usable for sterilization (see Patent Document 1 mentioned below).

Further, as disclosed in, for example, Patent Document 2 mentioned below, a technique has heretofore been known in which light emitted from a light source is guided by a light guide member including an optical fiber.

### PRIOR ART DOCUMENT

### Patent Documents

Patent Document 1: JP-A-2020-92968
Patent Document 2: JP-A-2004-026898

### SUMMARY OF THE INVENTION

### Problems to be Solved by the Invention

The structure disclosed in Patent Document 1 makes it possible to achieve such a small device that can be held by a user, and such a device can be used to sterilize a narrow area such as the inside of a shoe.

However, the device disclosed in Patent Document 1 has a structure in which a light extraction surface is provided in a housing to emit ultraviolet light, and therefore a relatively wide area is irradiated with ultraviolet light. Therefore, the device has room for improvement to inactivate microbes or viruses that may be present in a more local area by irradiating such an area with ultraviolet light.

Further, the technique disclosed in Patent Document 2 also has room for improvement because when this technique is used to irradiate a narrow area with light, there is a case where light in a light-emitting tube cannot efficiently be extracted.

In light of such problems, it is an object of the present invention to provide a device capable of irradiating a narrower area than ever before with ultraviolet light to inactivate microbes or viruses. It is also an object of the present invention to provide a discharge lamp capable of extracting a larger amount of light from a light-emitting tube.

### Means for Solving the Problems

The present invention is directed to a bacteria or viruses inactivation device including: a first light source that emits ultraviolet light having a principal wavelength band at least part of which is within a range of 200 nm or more and less than 240 nm; a housing body that houses the first light source; and a light guide body that has an elongated shape and guides the ultraviolet light emitted from the light source in a longitudinal direction, part of the light guide body, including a first end that is an end on a side close to the light source, being positioned in the housing body, wherein the light guide body is disposed in such a manner that a second end, which is an end on a side opposite to the first end, projects outside the housing body.

The “principal wavelength band” herein refers to a wavelength band across which an emission spectrum obtained by dispersing light according to wavelength to show intensity versus wavelength exhibiting light intensity equal to or more than 40% of the highest light intensity (peak intensity). Typically, the principal wavelength band includes the wavelength of peak intensity (peak wavelength).

The “inactivation” herein refers to a concept that includes killing at least some of bacteria or viruses or reducing their infectability. The “bacteria” herein refers to microorganisms such as bacteria and fungi (molds).

Ultraviolet light with a wavelength of 200 nm or more and less than 240 nm is known to have fewer negative effects on human bodies than ultraviolet light with a wavelength of 240 nm or more and less than 280 nm including ultraviolet light with a wavelength of 254 nm conventionally used as bactericidal rays. From the viewpoint of further reducing negative effects on human bodies, it is more preferred that at least part of the principal wavelength band of ultraviolet light emitted from the light source is within a range of 200 nm or more and less than 235 nm.

In the bacteria or viruses inactivation device, ultraviolet light emitted from the first light source is propagated through the elongated light guide body and emitted through the second end of the light guide body. This light guide body is positioned in such a manner that the second end, that is, the emission-side end thereof projects outside the housing body that houses the first light source. This makes it possible to irradiate a local area with ultraviolet light. For example, it is possible to inactivate bacteria or viruses that may be present in a very narrow space or on the surface of an article with a small occupied volume.

Further, the device described above is useful also when microbe or virus inactivation needs to be performed only on a specific area such as the surface of the skin of a human body.

The first light source that emits ultraviolet light having a principal wavelength band at least part of which is within a range of 200 nm or more and less than 240 nm may be any light source. A typical example of such a light source is an excimer lamp filled with a light-emitting gas containing KrCl or KrBr, an LED, or a laser diode. When the first light source is constituted from a lamp, the lamp is not limited to the above excimer lamp and may be a lamp using a material for wavelength conversion, such as a fluorescent material, to emit ultraviolet light having a principal wavelength band within a range of 200 nm or more and less than 240 nm.

It should be noted that in light of efficiently using ultraviolet light emitted from the first light source to inactivate bacteria or viruses, a light source suitable as the first light source is one that emits ultraviolet light having a principal wavelength band, the entirety of which is included in a range of 200 nm or more and less than 240 nm.

In the following description, the “bacteria or viruses inactivation device” may simply be abbreviated as “inactivation device”.

The light guide body may include an optical member that guides the ultraviolet light to the second end by internal total reflection.

Examples of such a member constituting the light guide body include glass rods, optical fibers, and light guides made of quartz, calcium fluoride, magnesium fluoride, or aluminum oxide (alumina, sapphire), and these members may be connected in series to each other.

The use of such a light guide body reduces the amount of ultraviolet light laterally radiated (leaked) to the outside during propagation of ultraviolet light in the light guide body toward the emission-side end (second end). This makes it possible to efficiently guide ultraviolet light to the second end.

The inactivation device may include an optical filter that is disposed at at least one position selected from among the first end, the second end, and an intermediate position between the first end and the second end of the light guide body to prevent travel of a wavelength component within a range of 240 nm or more and less than 280 nm that the ultraviolet light contains.

Even when, as the first light source, a light source that emits ultraviolet light having a principal wavelength band within a range of 200 nm or more and less than 240 nm is used, there is a case where ultraviolet light emitted from the first light source has a weak intensity within a wavelength range of 240 nm or more and less than 280 nm. In the case of a lamp that shows a steep emission spectrum, such as a KrCl excimer lamp or a KrBr excimer lamp, the emission spectrum may exhibit light intensity within a wavelength range of 240 nm or more and less than 280 nm, although it is much weaker than a peak intensity. Also when an LED is used as the first light source, the emission spectrum thereof may exhibit light intensity within a wavelength range of 240 nm or more and less than 280 nm.

As described above, there is a fear that irradiation with ultraviolet light with a wavelength of 240 nm or more and less than 280 nm may have negative effects on human bodies depending on irradiation time. The inactivation device includes a first light source that emits ultraviolet light having a principal wavelength band at least part of which is within a range of 200 nm or more and less than 240 nm, and therefore the amount of a component within a wavelength range of 240 nm or more and less than 280 nm contained in emitted ultraviolet light is relatively small. However, from the viewpoint of further reducing risk to human bodies, the intensity of ultraviolet light within such a wavelength range is preferably reduced as much as possible. Particularly, when the inactivation device is used for bacteria or viruses inactivation in a specific area such as the skin of a human body, it is conceivable that a human body is directly irradiated with ultraviolet light. In this situation where the inactivation device is used in such a way, it is important to reduce a component within a wavelength range of 240 nm or more and less than 280 nm contained in ultraviolet light as much as possible.

Since the inactivation device includes an optical filter that prevents the travel of a wavelength component within a range of 240 nm or more and less than 280 nm, it is possible to further prevent negative effects on human bodies.

The optical filter is preferably disposed at the first end or at an intermediate position between the first end and the second end. In this case, ultraviolet light that has passed through the optical filter propagates in the light guide body, which reduces the dose of ultraviolet light propagating in the light guide body. This makes it possible to retard the progress of deterioration of the light guide body. When the optical filter is disposed at the second end, the wavelength component of ultraviolet light whose travel should be prevented is reflected toward the first end side by the optical filter provided at the second end. In this case, deterioration of the light guide body may be progressed by ultraviolet light that travels in the light guide body from the second end toward the first end. Also from such a viewpoint, the optical filter is preferably disposed at the first end or at an intermediate position between the first end and the second end. This configuration is particularly effective when part of the light guide body is constituted from a light guide member containing a resin material, such as an optical fiber or a light guide, at a position closer to the second end than the optical filter.

Meanwhile, as described above, the first end of the light guide body is an end through which ultraviolet light from the first light source enters. If the optical filter is disposed in such a region, the dose itself of ultraviolet light taken into the light guide body may be reduced. Therefore, it can be said that the optical filter is particularly preferably disposed in an intermediate position between the first end and the second end from the viewpoints of taking as much as possible of ultraviolet light emitted from the first light source into the light guide body, preventing the progress of deterioration of the light guide body during propagation of ultraviolet light in the light guide body, and reducing as much as possible a component of a wavelength band having negative effects on human bodies from ultraviolet light emitted through the second end.

A method for disposing the optical filter at an intermediate position between the first end and the second end of the light guide body may be one in which a plurality of light guide members are connected in series to each other to form a light guide body, and the optical filter is disposed at any one of end surfaces excluding the incidence-side end surface of the light guide member positioned closest to the light source (referred to as “first light guide member” for convenience) and the emission-side end surface of the light guide member closest to the emission end (the second end of the light guide body) (referred to as “second light guide member” for convenience). For example, the optical filter may be disposed at the emission-side end surface of the first light guide member, that is, the end surface of the first light guide member connected to the subsequent light guide member or at the incidence-side end surface of the second light guide member, that is, the end surface of the second light guide member connected to the preceding light guide member. When another light guide member (referred to as “third light guide member” for convenience) is connected between the first light guide member and the second light guide member, in single or multiple series, the optical filter may be disposed at any one of the end surfaces of the third light guide member.

It should be noted that when the light guide body is formed by connecting a plurality of light guide members in series to each other, the optical filter is preferably provided at the end surface of one of the plurality of light guide members at least part of which is positioned inside the housing body. As an example, when the light guide body is formed by connecting the first light guide member and the second light guide member in series to each other, the optical filter is preferably provided at the end surface of the first light guide member. As another example, when one or more third light guide members are connected in series to each other between the first light guide member and the second light guide member, the optical filter is preferably provided at the end surface of the first light guide member or the end surface of the third light guide member part of which is positioned inside the housing body. This makes it possible to retard the progress of deterioration of part of the light guide body positioned outside the housing body (typically, the second light guide member).

The optical filter may be coated on the end surface of a member (light guide member) constituting the light guide body. That is, the light guide body may contain at least one light guide member whose end surface is coated with the optical filter. Such a configuration makes it possible to reduce the production cost of the inactivation device as compared to a configuration in which a substrate on which the optical filter is provided is brought into contact with the end surface of the light guide member.

One conceivable mode of the inactivation device is one in which the light guide body is formed by connecting a plurality of light guide members in series to each other, and one of the light guide members that is positioned closest to the emission-side end (second light guide member) is constituted from an optical fiber or a light guide. It is assumed that such an inactivation device will be used in a way where a user holds the second light guide member with their hand and adjusts the direction of irradiation to a target area.

When the second light guide member is constituted from an optical fiber or a light guide, ultraviolet light that has entered the second light guide member propagates toward the emission-side end (i.e., the second end of the light guide body) while being reflected in the second light guide member. However, when the user holds the second light guide member with their hand to adjust the irradiation direction in a narrow space, there may be a case where the second light guide member is bent at an acute angle or some of element wires are damaged or broken. In such case, if ultraviolet light propagating in the second light guide member contains a wavelength component within a range of 240 nm or more and less than 280 nm, the user may be irradiated with such an ultraviolet light laterally leaked from the second light guide member. Also from such a viewpoint, it can be said that the optical filter is preferably disposed at the first end of the light guide body or at an intermediate position between the first end and the second end of the light guide body. This makes it possible to significantly reduce a component within a wavelength range of 240 nm or more and less than 280 nm contained in ultraviolet light propagating in the second light guide member, thereby further reducing a risk that the user is irradiated with ultraviolet light within a wavelength range that may have negative effects on human bodies.

The second end of the light guide body may be convex outward.

For example, when the inactivation device is used to inactivate bacteria or viruses that may be present in a specific area on the skin of a human body, part of body fluid contained in the human body may be attached to the tip of the light guide body, that is, to the second end and its vicinity. Protein contained in the body fluid has the ability to absorb ultraviolet light within a range of 200 nm or more and less than 240 nm, and therefore if body fluid is attached to the second end of the light guide body, it is conceivable that the irradiance of ultraviolet light in a target area is reduced so that inactivation effect is reduced.

On the other hand, in the case of such a structure as described above in which the second end of the light guide body is convex outward, if coming into contact with body fluid, the second end is less likely to keep contact with the body fluid. This prevents a reduction in the irradiance of ultraviolet light. In this case, when the inactivation device is used by bringing the second end of the light guide body into contact with a human body during ultraviolet irradiation, from the viewpoint that the contact point on the human body is less likely to be physically damaged, it is preferred that the second end has a mildly curved convex shape and is typically constituted from a curved surface constituting part of a sphere or an oval sphere.

It should be noted that not only when the inactivation device is used for irradiation of a human body but also when the inactivation device is used under conditions where a liquid such as moisture may be present around it, it is possible to obtain an effect that the second end of the light guide body is less likely to keep contact with the liquid. For example, if moisture containing dirt or dust keeps attaching to the second end of the light guide body, irradiance reduction may be caused.

The inactivation device may include a flexible member that covers the second end of the light guide body and has permeability to the ultraviolet light.

In the case of such a configuration, when the inactivation device is used by bringing the emission-side end of the second light guide body into contact with a human body during ultraviolet irradiation, the contact point on the human body is less likely to be physically damaged. Examples of a material of such a flexible member include: a fluorine-based resin such as PTFE (polytetrafluoroethylene), PCTFE (polychlorotrifluoroethylene), ETFE (tetrafluoroethylene), PFA (perfluoroalkoxyalkane), PVDF (polyvinylidenefluoride), or FEP (perfluoroethylene-propene copolymer); PP (polypropylene); PE (polyethylene); PVA (polyvinyl alcohol); PVC (polyvinyl chloride); COC (cyclic olefin copolymer); and a silicone resin. Among these, PTFE can suitably be used in terms of availability. These raw materials can have the ability to transmit ultraviolet light when thicknesses thereof are extremely small and, on the other hand, can have the ability to reflect ultraviolet light when thicknesses thereof are large. Therefore, when a thin film-shaped flexible member made of PTFE or the like is disposed at the emission-side end surface (second end) of the light guide body, physical damage to a human body can be prevented while a reduction in the irradiance of ultraviolet light is prevented. For example, the outside surface of a region of about 1 mm to 5 mm, including the second end, in the light guide body may be covered with a thin film-shaped flexible member.

It should be noted that the “thin film” herein refers to a film having a thickness of 0.01 mm to 1.0 mm, preferably 0.02 mm to 0.5 mm.

Further, a region in the light guide body, from which ultraviolet light should not be emitted, may be covered with a flexible member thicker than that covering the second end by taking advantage of the property that the flexible member has the ability to reflect ultraviolet light when the thickness thereof is large.

The light guide body may include, at a region closer to the second end than the first end, a region whose outer diameter decreases toward the second end.

Such a configuration makes it easy to handle, in a narrow area, the light guide body positioned at the region closer to the second end. This makes it easy to irradiate a narrow area with ultraviolet light, which contributes to the inactivation of bacteria or viruses that may be present in the narrow area. As a typical example, when the light guide body includes a first light guide member positioned on the side close to the first light source (first end) and a second light guide member connected in series to the first light guide member and positioned close to the emission-side end (second end), a region in the second light guide member positioned close to the emission-side end surface (i.e., the second end) may have a shape whose outer diameter decreases toward the second end.

The inactivation device may include a light collection optical system that collects the ultraviolet light emitted from the first light source and guides it to the first end of the light guide body.

Such a configuration makes it possible to allow ultraviolet light from the first light source to efficiently enter the light guide body even when the incidence-side end surface (first end) of the light guide body and the light source are distant from each other.

The inactivation device may include a second light source that is housed in the housing body and has a principal wavelength band that is out of a range of 200 nm or more and less than 240 nm but is within at least one of a visible light range and an infrared range, wherein the light guide body guides light emitted from the second light source to the second end at a timing the same as or different from the ultraviolet light emitted from the first light source.

When a target area to be subjected to inactivation treatment is local, there may be a case where it is difficult to visually recognize the target area due to poor ambient light. When the inactivation device includes the second light source that emits light in a visible light range, an irradiation area can be irradiated with ultraviolet light while being lighted up with visible light when the inactivation device is used.

Depending on the type of bacteria or viruses, combination use of ultraviolet light and infrared light may enhance the inactivation effect. Therefore, when the inactivation device includes the second light source that emits light in an infrared range, the inactivation effect on a target area can be enhanced.

It should be noted that the second light source may include a light source that emits light in a visible light range and a light source that emits light in an infrared range.

The present invention is also directed to a treatment device including the bacteria or viruses inactivation device, wherein a treatment site is irradiated with the ultraviolet light emitted through the second end of the light guide body.

Such a configuration makes it possible to inactivate bacteria or viruses that may be present on the skin while performing predetermined treatment on a local treatment site, thereby making it possible to take measures to prevent infection. Examples of such a treatment device include endoscopes, dental cutting tools, and arthroscopes.

The bacteria or viruses inactivation device and the treatment device according to the present invention can provide sterilization and virus inactivation performance intrinsic to ultraviolet light without causing erythema or keratitis on the skin or eye of a human or an animal. This accords with Goal 3 “Ensure healthy lives and promote well-being for all at all ages” included in the Sustainable Development Goals (SDGs) led by the United Nations, and will greatly contribute to the goal target 3.3 “By 2030, end the epidemics of AIDS, tuberculosis, malaria and neglected tropical diseases and combat hepatitis, water-borne diseases and other communicable diseases”.

The present invention is also directed to a discharge lamp including: a light-emitting tube formed of a dielectric material and having an inside filled with a light-emitting gas; a first electrode disposed on a tube wall of the light-emitting tube; a second electrode disposed on the tube wall of the light-emitting tube at a position distant from the first electrode; and a light guide member part of which is connected to the tube wall of the light-emitting tube, wherein the light guide member includes a first end and a second end, which is on a side opposite to the first end and is positioned outside the light-emitting tube, and extends from its connection point with the tube wall of the light-emitting tube toward the second end in a direction getting away from the light-emitting tube.

In the case of this discharge lamp, since the light guide member is connected to the tube wall of the light-emitting tube, the light generated in the light-emitting tube is efficiently guided to the light guide member. Then, the light is propagated by the light guide member and then extracted to the outside through the second end of the light guide member positioned outside the light-emitting tube. Therefore, a larger amount of light generated in the light-emitting tube can be extracted as compared to, for example, a case where the light guide member is disposed distant from the light-emitting tube or the light guide member is connected to the light-emitting tube through another member.

The first end may be exposed to an internal space of the light-emitting tube.

The discharge lamp is formed by filling a light-emitting gas into the light-emitting tube formed of a dielectric material. When high voltage with a high frequency is applied to the first electrode and the second electrode disposed on the tube wall of the light-emitting tube, atoms or molecules (hereinafter simply referred to as “atoms or the like”) of the light-emitting gas are excited due to generation of discharge plasma, and therefore emitted light is obtained when they are returned to the ground state.

That is, discharge plasma is generated between the first electrode and the second electrode in the light-emitting tube, and therefore light is radiated mainly in a space sandwiched between the first electrode and the second electrode in the light-emitting tube (hereinafter referred to as “effective discharge space” for convenience). Therefore, the first end of the light guide member is preferably disposed as close to the effective discharge space as possible. In the case of such a configuration as described above, the first end of the light guide member can be brought close to the effective discharge space by exposing the light guide member to the inside of the light-emitting tube, which makes it possible to extract a larger amount of light from the light-emitting tube.

The light guide member may be disposed in such a manner the first end and the first electrode overlap each other in a normal direction of a wall surface of the light-emitting tube on which the first electrode is disposed.

Further, the present inventors have paid attention to the position of the first end of the light guide member exposed to the inside of the light-emitting tube and the position of the first electrode disposed on the tube wall of the light-emitting tube. That is, a larger amount of light can be extracted from the light-emitting tube by disposing the first end so as to overlap the effective discharge space in addition to by bringing the first end close to the effective discharge space.

The first end may be connected to the tube wall of the light-emitting tube, and a region, which faces the first end, in an inner wall of the light-emitting tube may overlap the first electrode in a normal direction of a wall surface of the light-emitting tube on which the first electrode is disposed.

When the first end of the light guide member is connected to the tube wall of the light-emitting tube, light generated in the light-emitting tube is guided to the light guide member through an inner wall facing the connection point of the first end. Therefore, a larger amount of light can be extracted from the light-emitting tube by disposing the inner wall so as to overlap the effective discharge space.

In the discharge lamp, the first electrode and the second electrode may be disposed distant from each other on a same wall surface of the light-emitting tube, and the light guide member and the light-emitting tube may be connected at a position between the first electrode and the second electrode in the wall surface of the light-emitting tube on which the first electrode and the second electrode are disposed.

As described above, the first end of the light guide member or an inner wall facing the point where the first end is connected is preferably disposed at a position overlapping the effective discharge space. Therefore, when the first electrode and the second electrode are disposed distant from each other on the same wall surface of the light-emitting tube, the light guide member may be connected at a position between the first electrode and the second electrode.

When the first end of the light guide member is exposed to the internal space of the light-emitting tube, the first end may be constituted from a curved surface convex toward the internal space. Typically, the curved surface may be part of a spherical surface or an oval spherical surface.

When the first end of the light guide member is constituted from such a curved surface as described above, the surface area of the first end can be made larger as compared to a case where the first end has a planar shape. This makes it possible to extract a larger amount of light from the light-emitting tube because the surface area of the first end, through which light generated in the light-emitting tube enters, is increased.

Further, from the viewpoint of preventing diffuse reflection at the incidence plane of light, the end surface of the first end of the light guide member may be mirror-polished.

In the discharge lamp, a dimension of an internal space of the light-emitting tube and a dimension of the first end of the light guide member may be almost the same in a direction in which the light guide member extends.

Such a configuration as described above makes it possible to increase the area of the first end of the light guide member through which light generated in the light-emitting tube enters or the area of an inner wall facing the point where the first end is connected. This is preferred because a light capture area can be increased. The phrase “a dimension of an internal space of the light-emitting tube and a dimension of the first end of the light guide member are almost the same” herein can mean that an error in dimension between them is 20% or less.

In the discharge lamp, the first electrode may be disposed continuously or discontinuously in an electrically connected state in regions, facing each other across an internal space of the light-emitting tube, in a wall surface of the light-emitting tube.

As described above, light is radiated due to the generation of discharge plasma in a space sandwiched between the first electrode and the second electrode in the light-emitting tube. Therefore, for example, when the first electrode is disposed in regions facing each other across the internal space of the light-emitting tube, discharge plasma can be generated entirely in the effective discharge space. When discharge plasma is generated entirely, a larger amount of light can be extracted through the first end of the light guide member or an inner wall facing the point where the first end is connected.

In the discharge lamp, a reflective layer whose reflectance to light generated by the light-emitting gas is higher than those of the first electrode and the second electrode may be provided at at least an interface between the tube wall of the light-emitting tube and the first electrode or an interface between the tube wall of the light-emitting tube and the second electrode.

Light generated in the light-emitting tube travels in various directions in the light-emitting tube. The first electrode and the second electrode have a certain reflectance to such light, but there is a case where the light reflectance of the surface of each of the electrodes is reduced depending on the wavelength of the light, the material and the processing accuracy of each of the electrodes, etc. On the other hand, when such a reflective layer as described above is provided at the interface between the tube wall of the light-emitting tube and the first electrode or the interface between the tube wall of the light-emitting tube and the second electrode, part of the light traveling toward each of the electrodes is efficiently reflected by the reflective layer so that a larger amount of light can be taken into the light guide member.

Such a reflective layer may be a sheet member formed of a metal such as aluminum. The reflective layer according to the above configuration is advantageous in that it can be achieved through a simple production process by sandwiching the above-described sheet member between the tube wall of the light-emitting tube and the electrode or by forming a reflective film on the surface of the electrode.

In the discharge lamp, the first electrode and the second electrode may be disposed distant from each other on a same wall surface of the light-emitting tube, and a reflective layer that reflects light generated by the light-emitting gas may be provided on the tube wall of the light-emitting tube at a position between the first electrode and the second electrode in the wall surface of the light-emitting tube on which the first electrode and the second electrode are disposed. It should be noted that the phrase “reflects light” means that the reflectance to incident light is 40% or more.

As described above, discharge plasma is formed in a space sandwiched between the first electrode and the second electrode, and therefore light is mainly generated at a position between both the electrodes. Therefore, the reflective layer is preferably provided on the tube wall of the light-emitting tube at such a position.

Such a reflective layer may be the above-described sheet member or a sheet member formed of a fluorine-based resin material such as polytetrafluoroethylene (PTFE). For example, a sheet member formed of PTFE may be wound around the light-emitting tube, or the light-emitting tube may be inserted into a tubular member formed of PTFE. As described above, the reflective layer according to the above configuration can be achieved through a simple production process.

It should be noted that as the reflective layer, a reflective film may be formed on the tube wall of the light-emitting tube. The reflective film may be, for example, a ceramic coating film containing silica particles or particles formed of a fluorine-based resin material. Alternatively, a dielectric multi-layer film obtained by laminating dielectric material layers different in refractive index may be used. The reflective film may be formed on either the outer wall or inner wall of the light-emitting tube.

The discharge lamp may be one that emits ultraviolet light having a principal wavelength band at least part of which is within a range of 200 nm or more and less than 240 nm. Typically, the discharge lamp may be an excimer lamp filled with a light-emitting gas containing KrCl or KrBr.

Such a configuration as described above makes it possible to extract a large amount of ultraviolet light from the light-emitting tube. This makes it possible to efficiently inactivate bacteria or viruses that may be present on the surface of an article and in a space by irradiating an irradiation target area with such ultraviolet light.

In the discharge lamp, the first electrode and the second electrode may be disposed distant from each other on a same wall surface of the light-emitting tube, the light guide member may be connected to the tube wall of the light-emitting tube at a position closer to the first electrode than the second electrode in a direction in which the first electrode and the second electrode are distant from each other, and the first electrode may be lower in electric potential than the second electrode in terms of absolute value.

As will be described later in detail, when the first electrode is configured to be lower in electric potential than the second electrode in terms of absolute value, the light guide member is preferably disposed on the side close to the first electrode. In this case, a larger amount of light can be extracted from the light-emitting tube as compared to a case where the light guide member is disposed on the side close to the second electrode.

The light guide member may be formed of a dielectric material.

### Effect of the Invention

The present invention makes it possible to inactivate bacteria or viruses in a narrower area than ever before. The discharge lamp according to the present invention makes it possible to extract a larger amount of light from the light-emitting tube.

### BRIEF DESCRIPTION OF THE DRAWINGS

FIG. 1 is a cross-sectional view schematically showing the structure of an inactivation device according to an embodiment of the present invention, in which some of components are expressed as block diagrams.

FIG. 2 is a schematic plan view when the inactivation device shown in FIG. 1 is viewed from its end projecting part side.

FIG. 3 is a cross-sectional view schematically showing a configuration example of an ultraviolet light source included in a light source unit.

FIG. 4 is a diagram schematically showing how ultraviolet light propagates in a first light guide member.

FIG. 5 is a cross-sectional view schematically showing the structure of the inactivation device including a second light guide member.

FIG. 6 is an enlarged view schematically showing the emission-side end of the second light guide member.

FIG. 7 is a further enlarged view schematically showing the emission-side end of the second light guide member.

FIG. 8 is a diagram schematically showing another configuration example of a light guide body.

FIG. 9 is a cross-sectional view schematically showing, in the same manner as FIG. 1, another configuration example of the inactivation device.

FIG. 10 is a cross-sectional view schematically showing another configuration example of the ultraviolet light source included in the light source unit.

FIG. 11 is a cross-sectional view schematically showing another configuration example of the light source unit.

FIG. 12 is a cross-sectional view schematically showing another configuration example of the light source unit.

FIG. 13 is a cross-sectional view schematically showing another configuration example of the light source unit.

FIG. 14 is a cross-sectional view schematically showing another configuration example of the light source unit.

FIG. 15 is a schematic diagram showing a configuration example of an endoscope including the inactivation device.

FIG. 16 is an enlarged view schematically showing the tip of an insertion part of the endoscope.

FIG. 17 is a diagram schematically showing another configuration example of the first light guide member.

FIG. 18 is a diagram schematically showing another configuration example of the second light guide member.

FIG. 19 is a cross-sectional view schematically showing another configuration example of the light source unit.

FIG. 20 is a cross-sectional view schematically showing another configuration example of the light source unit.

FIG. 21 is a diagram schematically showing another configuration example of the inactivation device.

FIG. 22A is a cross-sectional view schematically showing a first embodiment of a discharge lamp according to the present invention.

FIG. 22B is a plan view when the discharge lamp shown in FIG. 22A is viewed in a +X direction.

FIG. 23 is a conceptual diagram schematically showing discharge plasma generated between a first electrode and a second electrode.

FIG. 24A is a conceptual diagram showing an angular range of light taken into a second end of a light guide member.

FIG. 24B is a conceptual diagram of a case where the second end of the light guide member is brought closer to an effective discharge space than a case shown in FIG. 24A.

FIG. 24C is a conceptual diagram of a case where the second end of the light guide member is brought closer to the effective discharge space than a case shown in FIG. 24B so as to overlap the effective discharge space.

FIG. 25A is a conceptual diagram of an experimental system used for verification.

FIG. 25B is a conceptual diagram showing operation in verification.

FIG. 26 is a graph obtained by plotting the irradiance of light measured in verification.

FIG. 27A is a cross-sectional view when the first electrode and the second electrode are disposed not only on a −Z direction-side wall surface but also on a +Z direction-side wall surface of a light-emitting tube.

FIG. 27B is a plan view when the discharge lamp shown in FIG. 27A is viewed in the +X direction.

FIG. 28A is a conceptual diagram schematically showing an area where discharge plasma is mainly generated when the first electrode is disposed on the −Z direction-side wall surface of the light-emitting tube.

FIG. 28B is a conceptual diagram schematically showing an area where discharge plasma is mainly generated when the first electrode is disposed not only on the −Z direction-side wall surface of the light-emitting tube but also on the +Z direction-side wall surface of the light-emitting tube.

FIG. 28C is a conceptual diagram of a case where the first electrode is disposed so as to cover the entire circumference of the light-emitting tube.

FIG. 29 is a cross-sectional view showing a modification of the end surface of the second end.

FIG. 30 is a cross-sectional view showing a modification of the light guide member.

FIG. 31A is a cross-sectional view schematically showing a second embodiment of the discharge lamp according to the present invention.

FIG. 31B is a plan view of the discharge lamp shown in FIG. 31A when an incidence region is viewed from an internal space of the light-emitting tube in a −X direction.

FIG. 32 is a cross-sectional view showing a preferred configuration of the second embodiment.

FIG. 33A is a cross-sectional view showing the structure of another embodiment of the discharge lamp according to the present invention.

FIG. 33B is a cross-sectional view showing the structure of another embodiment of the discharge lamp according to the present invention.

FIG. 33C is a perspective view of the discharge lamp shown in FIG. 33B.

FIG. 33D is a perspective view conceptually showing one mode for forming a reflective layer on the light-emitting tube.

FIG. 34A is another cross-sectional view showing the structure of another embodiment of the discharge lamp according to the present invention.

FIG. 34B is a plan view when the discharge lamp shown in FIG. 34A is viewed in the −Z direction.

FIG. 35A is a cross-sectional view of a case where a plurality of light guide members are connected to each other.

FIG. 35B is a plan view when the discharge lamp shown in FIG. 35A is viewed in the −Z direction.

FIG. 36A is still another cross-sectional view showing the structure of another embodiment of the discharge lamp according to the present invention.

FIG. 36B is a plan view when the discharge lamp shown in FIG. 36A is viewed in an X direction.

FIG. 37A is still another cross-sectional view showing the structure of another embodiment of the discharge lamp according to the present invention.

FIG. 37B is a plan view when the discharge lamp shown in FIG. 37A is viewed in the X direction.

### MODE FOR CARRYING OUT THE INVENTION

### [First Configuration Example]

As a first configuration example of the present invention, embodiments of a bacteria or viruses inactivation device (hereinafter abbreviated as “inactivation device”) will be described with reference to the drawings as appropriate. It should be noted that the drawings disclosed herein merely show schematic illustrations, and the dimensional ratios on the drawings do not necessarily reflect the actual dimensional ratios. Further, the dimensional ratios are not necessarily the same between the drawings.

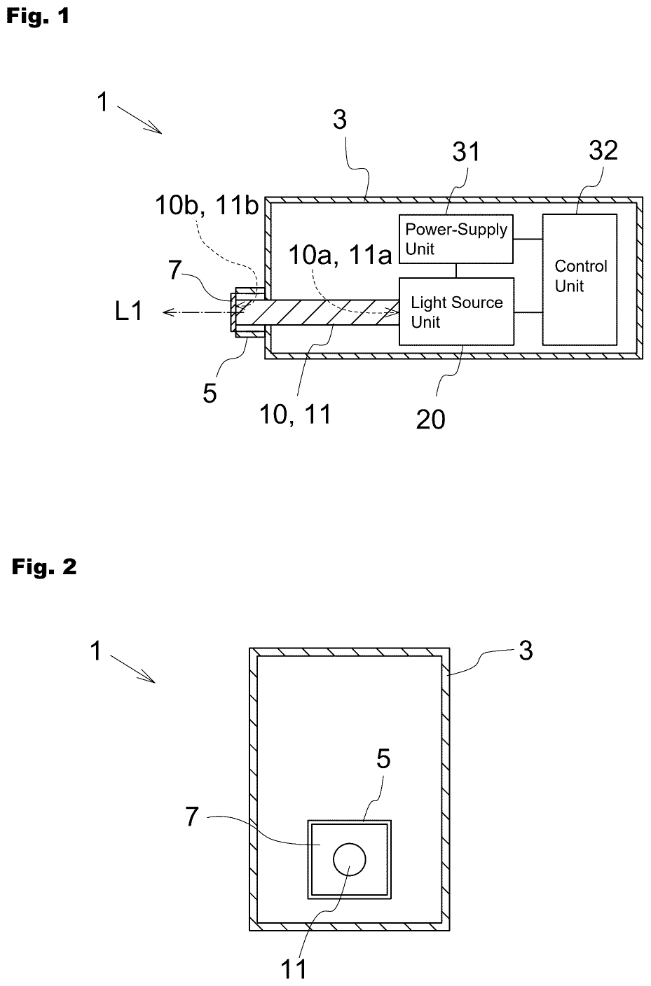

FIG. 1 is a cross-sectional view schematically showing the structure of an inactivation device according to an embodiment of the present invention, in which some of components are expressed as block diagrams. An inactivation device 1 includes a housing body 3 that houses a light source unit 20 and an end projecting part 5 provided on one of outside surfaces of the housing body 3. FIG. 2 is a schematic plan view when the inactivation device 1 is viewed from its end projecting part 5 side.

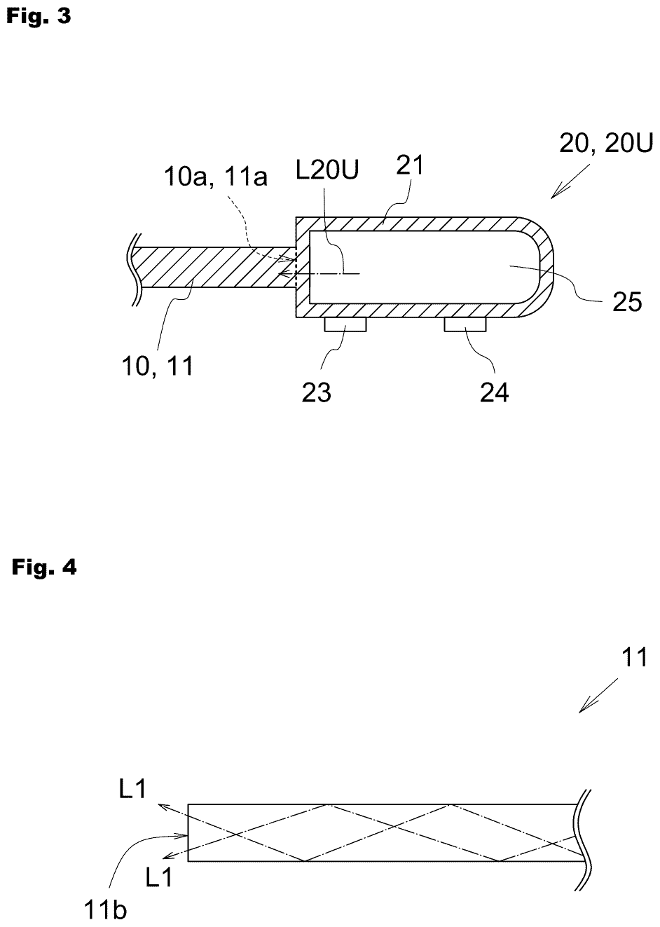

In the inactivation device 1, the housing body 3 houses the light source unit 20, a power-supply unit 31, and a control unit 32. As will be described later, the light source unit 20 includes an ultraviolet light source 20U (see FIG. 3) that emits ultraviolet light L1. The power-supply unit 31 is constituted from, for example, a power circuit including an inverter and others, and supplies electric power to the light source unit 20. The control unit 32 is a mechanism to control the power-supply unit 31 and controls the intensity of ultraviolet light L1 from the light source unit 20 and turning on/off of the light source unit 20.

The ultraviolet light source 20U (see FIG. 3) included in the light source unit 20 is a light source that emits ultraviolet light having a principal wavelength band at least part of which is within a range of 200 nm or more and less than 240 nm.

The end projecting part 5 projects outward from the outside surface of the housing body 3 and has a tubular shape surrounding a light guide body. The end projecting part 5 may be formed of the same material as the housing body 3. The housing body 3 is preferably formed of a material having resistance to ultraviolet light. For example, the housing body 3 is formed of a resin such as PTFE or a metal such as stainless steel or aluminum.

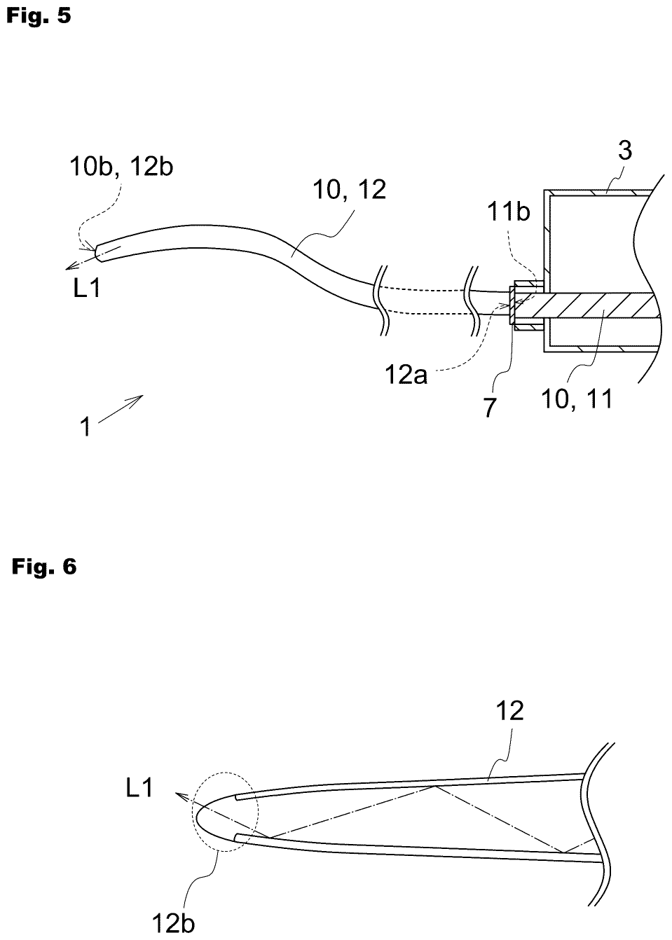

As shown in FIG. 1, the inactivation device 1 includes a light guide body 10 for guiding ultraviolet light emitted from the light source unit 20 to the end projecting part 5 side. FIG. 1 shows, as an example, a case where the light guide body 10 is constituted from a single first light guide member 11. The light guide body 10 is preferably configured to guide ultraviolet light emitted from the light source unit 20 to the end projecting part 5 side by repeated total internal reflection. Typical examples of the light guide body 10 include glass rods, optical fibers, light guides formed of quartz, calcium fluoride, magnesium fluoride, aluminum oxide (alumina, sapphire), or the like. It should be noted that FIG. 1 shows, as an example, a case where the light guide body 10 included in the inactivation device 1 is constituted from a single first light guide member 11, but the light guide body 10 may be constituted from a plurality of light guide members connected in series to each other. This point will be described later with reference to FIG. 5 and others.

More specifically, as shown in FIG. 1, the light guide body 10 has an elongated shape, and part of the light guide body 10, including a first end 10a on the side close to the light source unit 20, is positioned inside the housing body 3. On the other hand, a second end 10b of the light guide body 10 on the side opposite to the first end 10a is positioned outside the housing body 3. In the present embodiment, from the viewpoint of preventing attachment of foreign matter such as dust, the second end 10b-side circumference of the light guide body 10 is covered with the end projecting part 5. It should be noted that the end surface of the second end 10b of the light guide body 10 is not covered with the end projecting part 5. However, a configuration in which the second end 10b-side part of the light guide body 10 is not covered with the end projecting part 5 is also within the scope of the present invention.

It should be noted that in the case of the inactivation device 1 shown in FIG. 1, since the light guide body 10 is constituted from the first light guide member 11, the first end 10a, which is the light source unit 20-side end of the light guide body 10, corresponds to an incidence-side end 11a of the first light guide member 11, and the second end 10b of the light guide body 10, which is the end on the side opposite to the light source unit 20, corresponds to an emission-side end 11b of the first light guide member 11.

In the inactivation device 1 of the present embodiment, an optical filter 7 is provided at the emission-side end 11b of the first light guide member 11. This optical filter 7 serves the function of substantially transmitting light within a wavelength range of 200 nm or more and less than 240 nm but preventing the travel of light within a wavelength range of 240 nm or more and less than 280 nm.

The optical filter 7 may be a dielectric multi-layer film obtained by laminating layers different in refractive index. For example, the optical filter is a dielectric multi-layer film obtained by laminating a silica (SiO₂) layer and a hafnia (HfO₂) layer different in refractive index. Alumina (Al₂O₃), zirconia (ZrO₂), and the like can be used as other materials.

The film thickness of each layer and the number of layers of the dielectric multi-layer film are adjusted so that the optical filter 7 substantially transmits ultraviolet light within a wavelength range of 200 nm or more and less than 240 nm and prevents the travel of ultraviolet light within a wavelength range of 240 nm or more and less than 300 nm. The optical filter 7 is more preferably configured to substantially transmit ultraviolet light within a wavelength range of 200 nm or more and less than 235 nm, and is particularly preferably configured to substantially transmit ultraviolet light within a wavelength range of 200 nm or more and less than 230 nm.

It should be noted that the phrase “the optical filter 7 substantially transmits ultraviolet light within a wavelength range of 200 nm or more and less than 240 nm” means that when ultraviolet light enters the optical filter 7 at right angle, that is, at an incident angle of 0°, the maximum transmittance of ultraviolet light within a wavelength range of 200 nm or more and less than 240 nm is 20% or more. It should be noted that when ultraviolet light enters the optical filter 7 at an incident angle of 0°, the maximum transmittance of ultraviolet light within a wavelength range of 200 nm or more and less than 240 nm is more preferably 30% or more, particularly preferably 40% or more. The same applies to the other wavelength ranges.

The phrase “the optical filter 7 prevents the travel of ultraviolet light within a wavelength range of 240 nm or more and less than 300 nm” means that after ultraviolet light enters the optical filter 7, the ratio of the intensity of light within a wavelength range of 240 nm or more and less than 300 nm to the intensity (peak intensity) of light at a peak wavelength within a range of 200 nm or more and less than 240 nm is made lower than that before entry to the optical filter 7. For example, the intensity of light within a range of 240 nm or more and less than 300 nm in ultraviolet light L1 that has passed through the optical filter 7 is preferably reduced to less than 5%, more preferably less than 3%, particularly preferably less than 1% of the peak intensity.

That is, when the optical filter 7 is provided in the light guide body 10, the intensity of a component within a wavelength range of 240 nm or more and less than 300 nm, which may have negative effects on human bodies, in ultraviolet light L1 emitted from the inactivation device 1 through the light guide body 10 is sufficiently reduced. However, when the intensity of light within a wavelength range of 240 nm or more and less than 300 nm in the spectrum of ultraviolet light emitted from the light source unit 20 is extremely low to the extent that there is no need to consider negative effects on human bodies, it is not always necessary to provide the optical filter 7 in the light guide body 10. For example, when the light source unit 20 includes an optical member that serves the same function as the optical filter 7, it is not always necessary to provide the optical filter 7 at the end surface of the light guide body 10.

The installation position of the optical filter 7 can appropriately be adjusted according to the mode of a light guide member constituting the light guide body 10. This point will be described later.

FIG. 3 is a cross-sectional view schematically showing a configuration example of the ultraviolet light source 20U included in the light source unit 20. FIG. 3 shows a case where the ultraviolet light source 20U is constituted from an excimer lamp. The ultraviolet light source 20U corresponds to the “first light source”.

The ultraviolet light source 20U has a light-emitting tube 21 formed of a dielectric material such as quartz and a pair of electrodes 23, 24 disposed on the outer surface of the tube wall of the light-emitting tube 21. In the inside of the light-emitting tube 21, a light-emitting space 25 is formed which is filled with a light-emitting gas containing, for example, KrCl. The pair of electrodes 23 and 24 are disposed distant from each other, and the electric voltage is supplied to them through the power-supply unit 31 (FIG. 1). When applied to the pair of electrodes 23 and 24, the electric voltage is applied to the light-emitting gas in the light-emitting space 25 through the dielectric material so that dielectric-barrier discharge occurs and ultraviolet light L20U is generated by excimer emission. When the light-emitting gas contains KrCl, ultraviolet light L20U exhibits a spectrum having a peak wavelength at around 222 nm. It should be noted that the expression “around” herein means that there is a case where an acceptable error of about 1 nm to 5 nm is caused depending on the mixing ratio of the gas filled in the light-emitting space 25 or individual difference.

The light-emitting gas filled in the light-emitting space 25 is not limited as long as it is a material that can generate ultraviolet light L20U having a principal wavelength band at least part of which is within a range of 200 nm or more and less than 240 nm. An example of such a material other than KrCl is KrBr.

In the ultraviolet light source 20U shown in FIG. 3, the first light guide member 11 constituting the light guide body 10 is connected to part of the outer wall of the light-emitting tube 21. In this case, the first end of the light guide body 10, that is, the incidence-side end 11a of the first light guide member 11 is in contact with the outer wall of the light-emitting tube 21. From the viewpoint of ease of production, the light-emitting tube 21 and the first light guide member 11 are preferably formed of the same material. In this case, the light-emitting tube 21 and the first light guide member 11 are integrally formed.

Ultraviolet light L20U generated in the light-emitting space 25 enters the light guide body 10 (first light guide member 11) and propagates in the light guide body 10 (first light guide member 11). Since the first light guide member 11 is formed of quartz or the like, the ultraviolet light L20U propagates in the first light guide member 11 by repeated total reflection caused by the difference in refractive index between the constituent material of the first light guide member 11 and air. Then, the ultraviolet light L20U reaches the emission-side end 11b of the first light guide member 11, that is, the second end 10b of the light guide body 10, and is then emitted outside as ultraviolet light L1 (see FIG. 1, FIG. 4).

When the ultraviolet light source 20U has a structure shown in FIG. 3, the ultraviolet light source 20U may have a reflective member for guiding ultraviolet light L20U traveling toward the side opposite to the first light guide member 11 to the first light guide member 11 side so that ultraviolet light L20U generated in the light-emitting space 25 efficiently enters the first light guide member 11.

The inactivation device 1 shown in FIG. 1 emits, through the end (second end 10b) of the light guide body 10 having an elongated shape, ultraviolet light L1 having a principal wavelength band at least part of which is within a range of 200 nm or more and less than 240 nm which is effective at inactivating bacteria or viruses. This contributes to inactivation treatment performed on a narrower area than ever before.

It should be noted that the area of the cross-section of the first light guide member 11 taken along a plane perpendicular to the longitudinal direction (axial direction) thereof is preferably 1 mm² to 100 mm², more preferably 10 mm² to 20 mm².

As shown in FIG. 5, the light guide body 10 included in the inactivation device 1 may be formed by connecting a plurality of light guide members 11 and 12 to each other. A second light guide member 12 is typically an optical fiber or a light guide obtained by coating an optical fiber with a predetermined coating material, and has flexibility. It should be noted that the phrase “has flexibility” means that the shape or direction of the second light guide member 12 can easily be changed by a user who holds it.

In the case of the mode shown in FIG. 5, the light guide body 10 is formed by connecting the first light guide member 11 and the second light guide member 12 in series to each other, the incidence-side end 11a of the first light guide member 11 closest to the light source unit 20 (FIG. 1) corresponds to the first end 10a of the light guide body 10, and the emission-side end 11b of the second light guide member 12 closest to the emission side through which ultraviolet light L1 is extracted corresponds to the second end 10b of the light guide body 10. The optical filter 7 is disposed at the boundary between the first light guide member 11 and the second light guide member 12 which is an intermediate position between the first end 10a and the second end 10b of the light guide body 10.

Such a configuration makes it possible to locally irradiate a narrower area or a recessed portion with ultraviolet light L1, which contributes to bacteria or viruses inactivation treatment performed on a local area.

When the second light guide member 12 is constituted from an optical fiber or a light guide, the surface thereof is often coated with a resin or the like. When the optical filter 7 is provided at the boundary between the first light guide member 11 and the second light guide member 12, the intensity of a wavelength component within a range of 240 nm or more and less than 280 nm in ultraviolet light propagating in the second light guide member 12 is significantly reduced. This makes it possible to prevent the progress of deterioration of the second light guide member 12 due to a reduced dose of ultraviolet light propagating in the second light guide member 12.

As shown in FIG. 6, an emission-side end 12b of the second light guide member 12 is preferably convex outward. Such a configuration makes, when the inactivation device 1 is used to inactivate bacteria or viruses that may be present on the surface of the skin of a specific area in a human body, body fluid less likely to keep attaching to the emission-side end 12b even when attached thereto. Protein contained in body fluid has the ability to absorb ultraviolet light within a range of 200 nm or more and less than 240 nm, and therefore if body fluid keeps attaching to the emission-side end 12b of the second light guide member 12, there is a fear that the irradiance of ultraviolet light L1 on an irradiated surface is reduced. From the viewpoint of efficiently performing inactivation treatment, it is important that the emission-side end 12b of the second light guide member 12 (which also corresponds to the second end 10b of the light guide body 10) constituting the end through which ultraviolet light L1 is emitted is configured to be less likely to keep contact with body fluid.

It should be noted that using the structure shown in FIG. 6 for the second light guide member 12 is effective not only when inactivation treatment is performed on the skin of a human body but also when inactivation treatment is performed on a narrow area in an environment where moisture is present around it.

From the viewpoint of irradiating a local area with ultraviolet light L1, as shown in FIG. 6, part of the second light guide member 12 close to the emission-side end 12b preferably has a shape (tapered shape) whose outer diameter decreases toward the emission-side end 12b.

As shown in FIG. 7, the emission-side end 12b of the second light guide member 12 may be covered with a thin film-shaped flexible member 15. Particularly, in a situation where the skin of a specific area in a human body is irradiated with ultraviolet light L1, it is conceivable that the emission-side end 12b of the second light guide member 12 comes into contact with the skin. When the emission-side end 12b of the second light guide member 12 is covered with the flexible member 15, it is possible to obtain an effect that the skin is less likely to be physically damaged.

Examples of the material of the flexible member 15 include various resins such as PTFE, ETFE, PFA, PVDF, PP, PE, PVA, PVC, COC, and silicone resins. The thickness of the flexible member 15 positioned at the emission-side end 12b is preferably 0.01 mm to 1.0 mm, more preferably 0.02 mm to 0.5 mm. The material described above can have the ability to transmit ultraviolet light L1 when the thickness thereof is extremely small, which makes it possible to reduce physical damage to a target such as a human body while preventing irradiance reduction. Further, when provided, the flexible member 15 serves the function of diffusing and transmitting ultraviolet light L1. This makes it possible to, for example, collectively irradiate, with ultraviolet light, almost the entirety of a local area as a target of inactivation treatment.

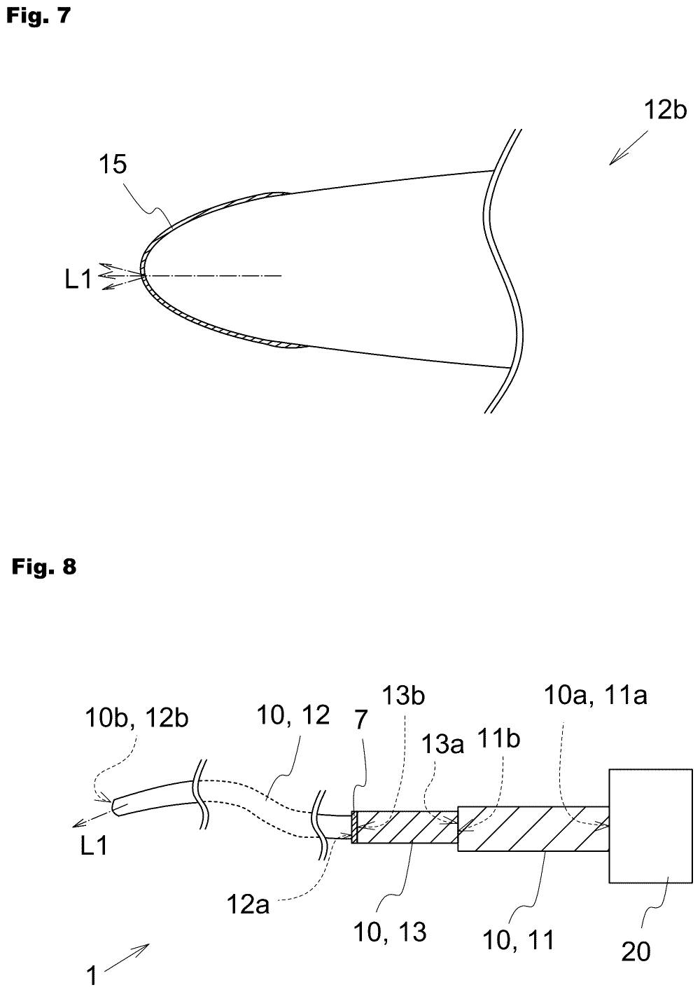

As shown in FIG. 8, the light guide body 10 may include, in addition to the first light guide member 11 disposed at a position closest to the light source unit 20 and the second light guide member 12 disposed at a position closest to the end through which ultraviolet light L1 is emitted, a third light guide member 13 disposed between these light guide members 11, 12. In this case, the third light guide member 13 may be formed by connecting a plurality of light guide members in series to each other. In other words, the light guide body 10 may be formed by connecting three or more light guide members in series to each other. In the case shown in FIG. 8, the optical filter 7 is disposed at the boundary between the third light guide member 13 and the second light guide member 12, i.e., at an incidence-side end 12a of the second light guide member 12 or an emission-side end 13b of the third light guide member 13. This position corresponds to the intermediate position between the first end 10a and the second end 10b of the light guide body 10.

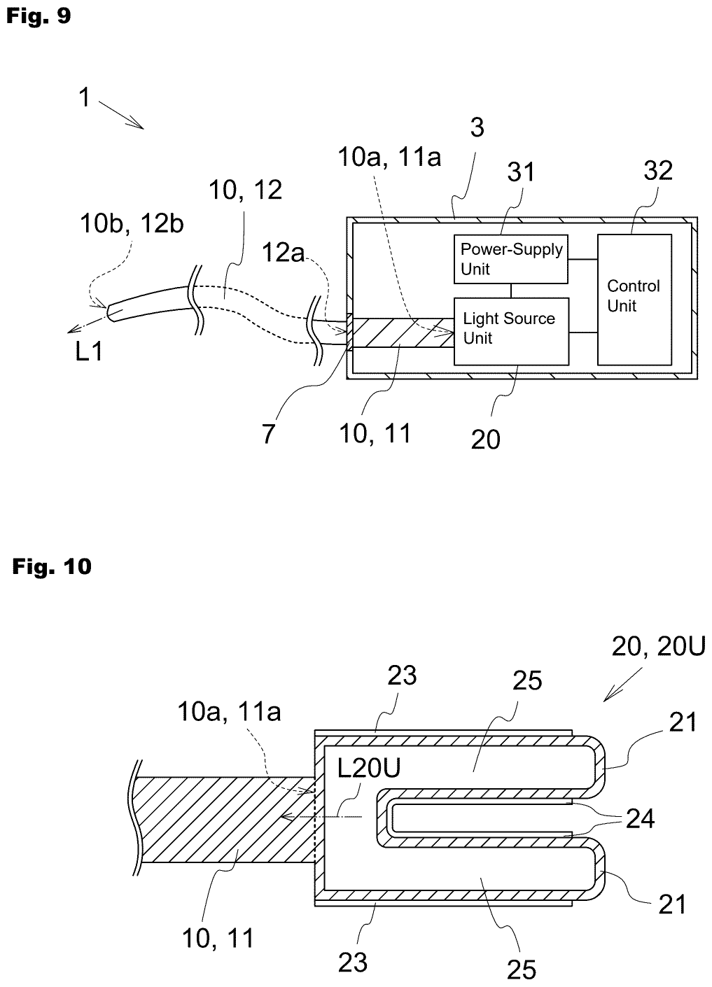

As described above, the light guide body 10 included in the inactivation device 1 is not limited as long as the second end 10b that is the end on the side opposite to the light source unit 20 projects outward from the housing body 3. Therefore, when the light guide body 10 is formed by connecting a plurality of light guide members (11, 12, . . . ) in series to each other, it is not always necessary to provide the end projecting part 5 on one of the outside surfaces of the housing body 3 (see FIG. 9). In the case of the inactivation device 1 shown in FIG. 9, the first light guide member 11 is positioned inside the housing body 3, but the second light guide member 12 connected in series to the first light guide member 11 projects outward from the housing body 3. It should be noted that as described above with reference to FIG. 8, the same discussion can be applied also to the case where the light guide body 10 includes three or more light guide members.

The structure of the ultraviolet light source 20U included in the light source unit 20 is not limited to the example shown in FIG. 3. FIG. 10 is a cross-sectional view schematically showing the structure of a different mode of the ultraviolet light source 20U shown in FIG. 3.

In the ultraviolet light source 20U shown in FIG. 10, the electrode 23 and the electrode 24 are disposed on the outside tube wall and the inside tube wall of the light-emitting tube 21 having a U shape, respectively. In the inside of the light-emitting tube 21, the light-emitting space 25 is formed which is filled with a light-emitting gas. Part of the wall surface of the light-emitting tube 21 is in contact with the first end 10a of the light guide body 10 (more specifically, the incidence-side end 11a of the first light guide member 11) so that the light-emitting tube 21 and the light guide body 10 are connected to each other.

Also in the case of the configuration shown in FIG. 10, ultraviolet light L20U derived from excimer light generated in the light-emitting space 25 by applying electric voltage between the electrodes 23, 24 enters the light guide body 10 and propagates in the light guide body 10 toward the emission-side end (second end 10b) so that ultraviolet light L1 is extracted outside through the second end 10b. As described above with reference to FIG. 5 and FIG. 8, the same applies to the case where the light guide body 10 includes another light guide member such as the second light guide member 12.

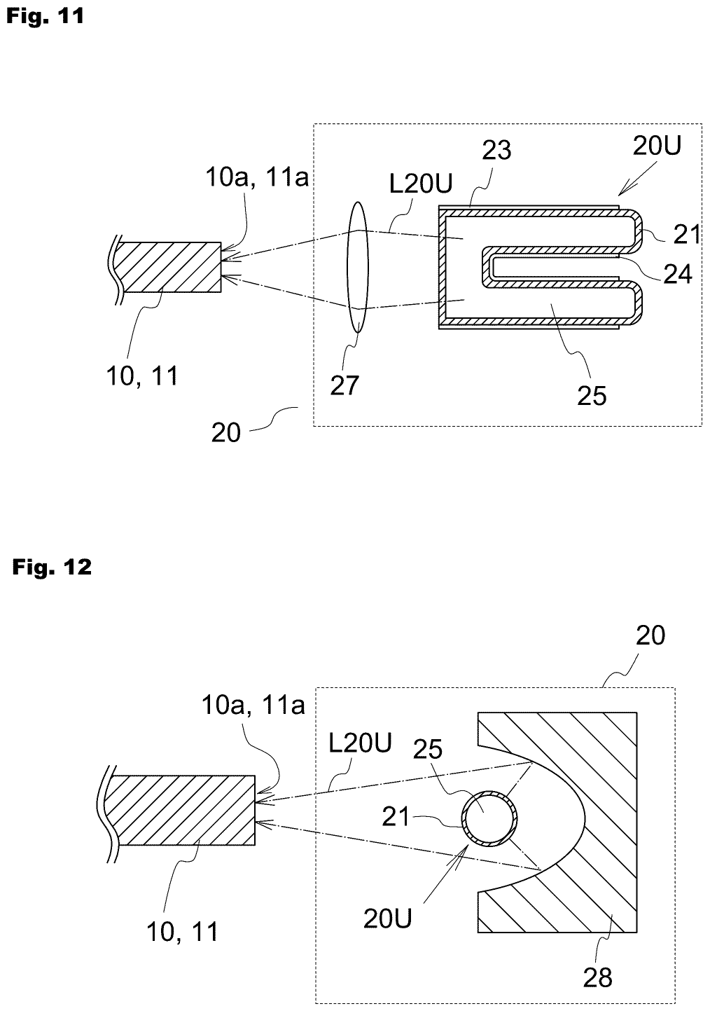

As shown in FIG. 11, the inactivation device 1 may include a condenser lens 27 for guiding ultraviolet light L20U generated by the ultraviolet light source 20U to the first end 10a of the light guide body 10. Alternatively, as shown in FIG. 12, the inactivation device 1 may include a condensing reflector 28 for guiding ultraviolet light L20U generated by the ultraviolet light source 20U to the first end 10a of the light guide body 10. That is, in the present invention, the light guide body 10 and the ultraviolet light source 20U do not always need to be in contact with each other. These condenser lens 27 and the condensing reflector 28 correspond to the “light collection optical system”. Typically, the condenser lens 27 is, for example, a convex lens, and the condensing reflector 28 is, for example, an ellipsoid mirror.

Further, as shown in FIG. 3, FIG. 10, and FIG. 11, when the light-emitting tube 21 (sealed body) of the ultraviolet light source 20U has an elongated shape, the surface (light radiation surface) through which ultraviolet light L20U generated in the light-emitting space 25 is extracted is preferably formed on the one end side of the light-emitting tube 21. More specifically, it is preferred that the first end 10a of the light guide body 10 is connected to the radiation surface or disposed to face the radiation surface. When the light radiation surface is formed on the one end side of the sealed body, the light-emitting space 25 in the sealed body can easily have depth so that ultraviolet light L20U can be extracted at a high radiant intensity through the light radiation surface toward the light guide body 10.

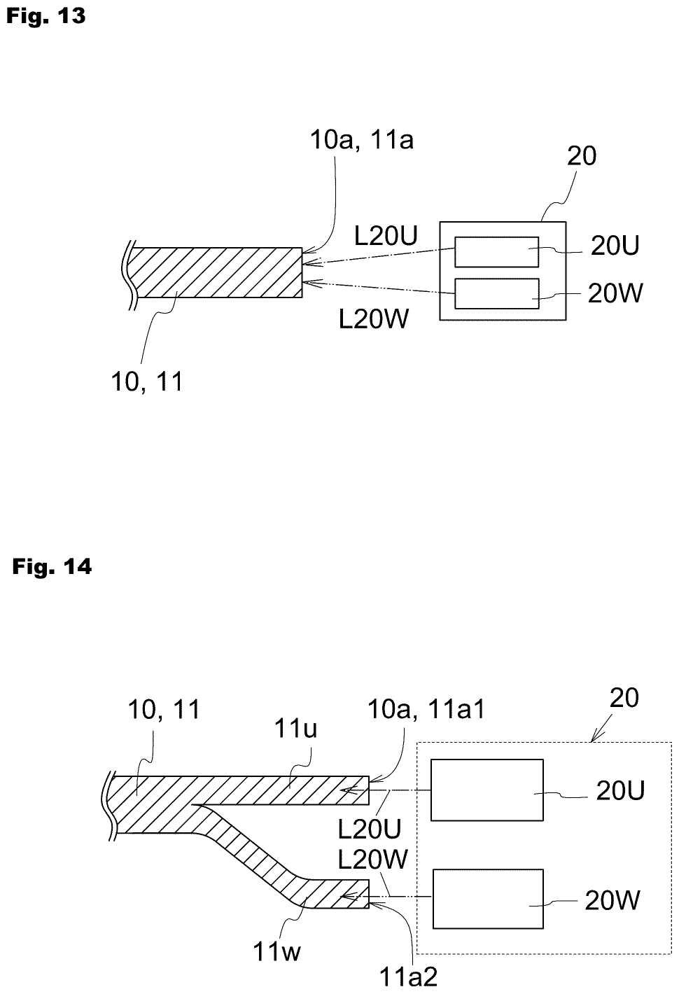

As shown in FIG. 13 and FIG. 14, the light source unit 20 may include, in addition to the ultraviolet light source 20U, a visible light source 20W. The visible light source 20W is typically an LED or a lamp that emits white light, but is not limited to a white light source as long as it is a light source that emits light in a visible light range. In this case, the visible light source 20W corresponds to the “second light source”.

In the case shown in FIG. 13, ultraviolet L20U from the ultraviolet light source 20U and visible light L20W from the visible light source 20W enter the first end 10a of the light guide body 10, more specifically, the incidence-side end 11a of the first light guide member 11. Both the ultraviolet light L20U and the visible light L20W propagate in a mixed state in the first light guide member 11 and are guided to the second end 10b of the light guide body 10.

In the case shown in FIG. 14, the first light guide member 11 is branched into a first branch 11u and a second branch 11w on its light source unit 20 side. An incidence-side end 11al of the first branch 11u corresponds to the first end 10a of the light guide body 10, and ultraviolet light L20U from the ultraviolet light source 20U enters the first branch 11u. On the other hand, visible light L20W from the visible light source 20W enters an incidence-side end 11a2 of the second branch 11w. Also in this case, both the ultraviolet light L20U and the visible light L20W are mixed in the middle of the first light guide member 11, and such mixed light propagates in the first light guide member 11 and is guided to the second end 10b of the light guide body 10.

When a target area to be subjected to inactivation treatment is local, it is conceivable that the target area is difficult to visually be recognized due to poor ambient light. However, such a configuration as described above makes it possible to irradiate an irradiation area with ultraviolet light L1 while lighting up the irradiation area with visible light because when the inactivation device 1 is used, not only ultraviolet light L1 but also visible light is emitted. It should be noted that in the case shown in FIG. 14, the first light guide member 11 is branched, but another light guide member for propagating visible light L20W may be provided in parallel to the first light guide member 11 to guide visible light L20W to the end projecting part 5 (see FIG. 1). Further, when the light source unit 20 includes the ultraviolet light source 20U and the visible light source 20W, the ultraviolet light source 20U and the visible light source 20W do not always need to be turned on at the same time and may be turned on at different timings from each other.

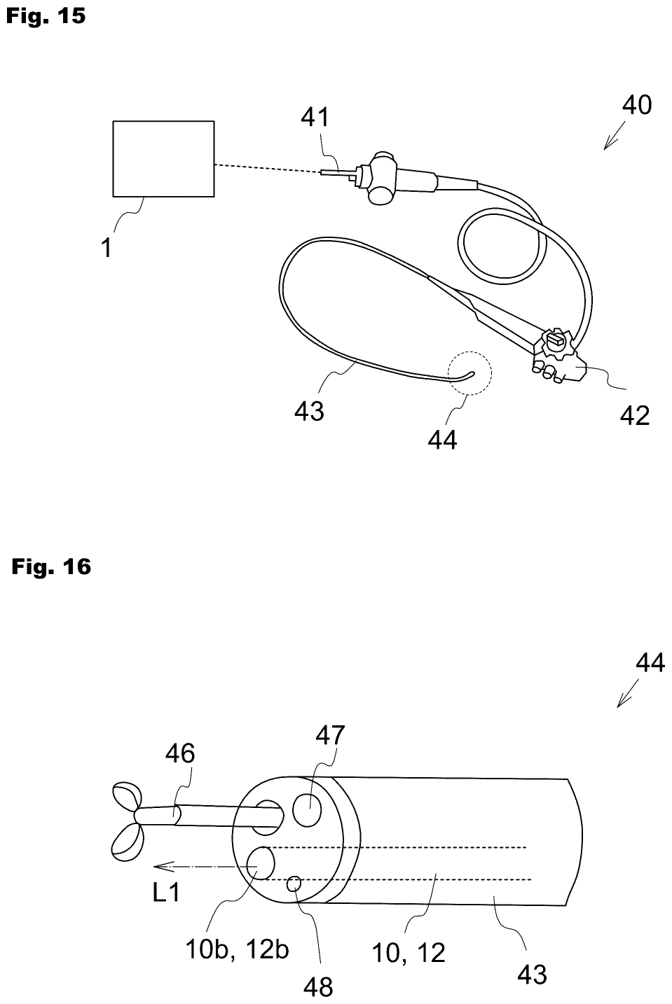

FIG. 15 shows an example of an endoscope 40 including the inactivation device 1. The endoscope 40 includes a connector 41, an operating part 42, and an insertion part 43. The connector 41 is connected to a system main body including the inactivation device 1. The operating part 42 typically includes an angle knob to control the curvature of the endoscope in vertical and horizontal directions, an air and water supply button, a suction button, and a forceps port through which a treatment tool is inserted. The insertion part 43 is a cable for an endoscope.

FIG. 16 is an enlarged view schematically showing the tip of the insertion part 43 shown in FIG. 15. The insertion part 43 houses, in addition to a treatment tool 46 for performing predetermined treatment on the tissues of a human body, an objective lens 47, and a suction port 48 for sucking collected tissues or foreign matter, the second light guide member 12 described above. The endoscope 40 shown in FIG. 15 and FIG. 16 makes it possible to perform microbe or virus inactivation treatment on the surface of a specific treatment site while observing the inside of an organ because the treatment site can be irradiated with ultraviolet light L1 emitted through the emission-side end 12b of the second light guide member 12, i.e., the second end 10b of the light guide body 10.

The endoscope shown in FIG. 15 and FIG. 16 is an example of a treatment device. Other examples of the treatment device including the inactivation device 1 include dental cutting tools and arthroscopes.

### OTHER EMBODIMENTS

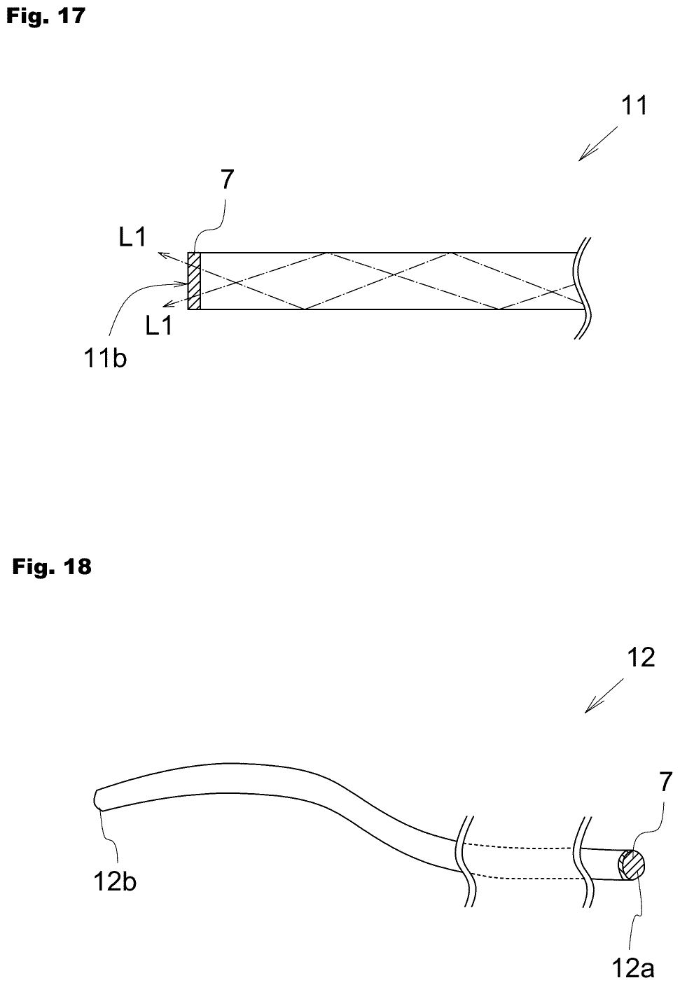

Other embodiments of the inactivation device 1 will be described below.
<1> The optical filter 7 may be coated on the end surface of a light guide member constituting the light guide body 10. For example, as shown in FIG. 17, the optical filter 7 may be coated on the emission-side end 11b of the first light guide member 11 constituting the light guide body 10. Alternatively, when the light guide body 10 includes the first light guide member 11 and the second light guide member 12, for example, as shown in FIG. 18, the optical filter 7 may be coated on the incidence-side end 12a of the second light guide member 12. The optical filter 7 may be coated on at least one of the incidence-side end 11a of the first light guide member 11 and the emission-side end 12b of the second light guide member 12.

However, from the viewpoint of taking a large amount of ultraviolet light L20U from the light source unit 20 into the first light guide member 11, it is preferred that the optical filter 7 is not provided at the incidence-side end 11a of the first light guide member 11. In other words, it is preferred that the optical filter 7 is provided at at least one position selected from among the emission-side end 11b of the first light guide member 11, the incidence-side end 12a of the second light guide member 12, and the emission-side end 12b of the second light guide member 12.

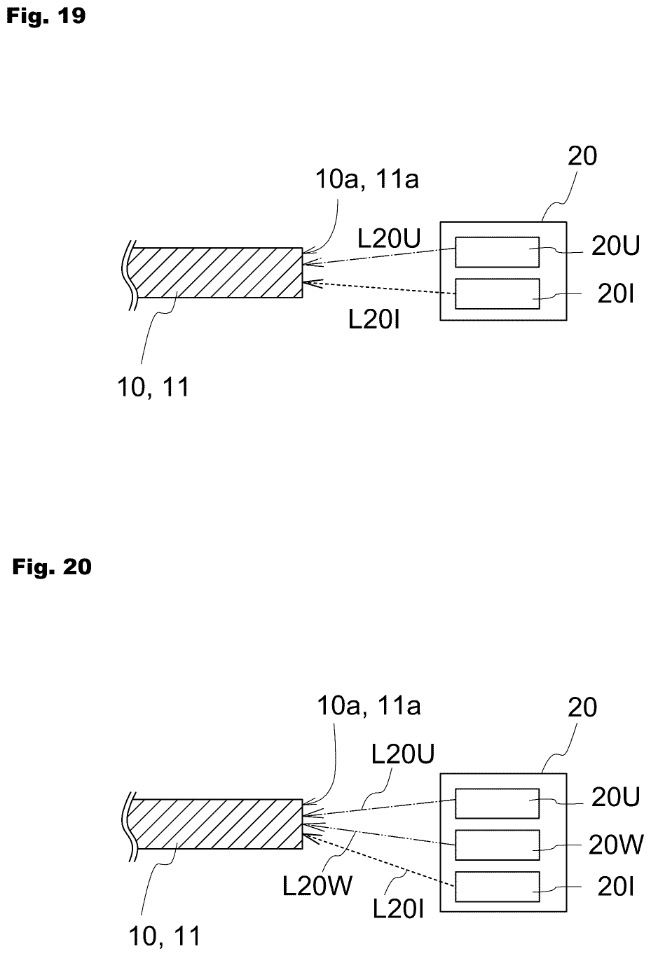

As shown in FIG. 8, when the light guide body 10 is formed by connecting the first light guide member 11, the third light guide member 13, and the second light guide member 12 in series to each other, the optical filter 7 may be coated on at least one selected from among the emission-side end 11b of the first light guide member 11, an incidence-side end 13a of the third light guide member 13, the emission-side end 13b of the third light guide member 13, and the incidence-side end 12a of the second light guide member 12.
<2> As shown in FIG. 19, the light source unit 20 may include, in addition to the ultraviolet light source 20U, an infrared light source 20I. The infrared light source 20I is a light source that emits, for example, infrared light L20I having a principal wavelength band within an infrared range of 700 nm to 2000 nm. It should be noted that in this case, as in the case shown in FIG. 14, the first light guide member 11 may have a plurality of branches at its incidence-side end so that lights from the respective light sources enter these branches, respectively.

Depending on the type of bacteria or viruses that may be present in an irradiation target point, inactivating action may be enhanced by irradiation with infrared light L20I in addition to ultraviolet light L20U due to the so-called hurdle effect. Such a configuration as described above makes it possible to enhance the inactivation effect because not only ultraviolet light but also infrared light is emitted through the second end 10b of the light guide body 10. In this configuration, the infrared light source 20I corresponds to the “second light source”.

It should be noted that the ultraviolet light source 20U and the infrared light source 20I may be turned on at the same time or at different timings. In other words, mixed light of ultraviolet light L1 and infrared light L20I may be emitted through the second end 10b of the light guide body 10, or ultraviolet light L1 and infrared light L20I may be emitted at different timings.

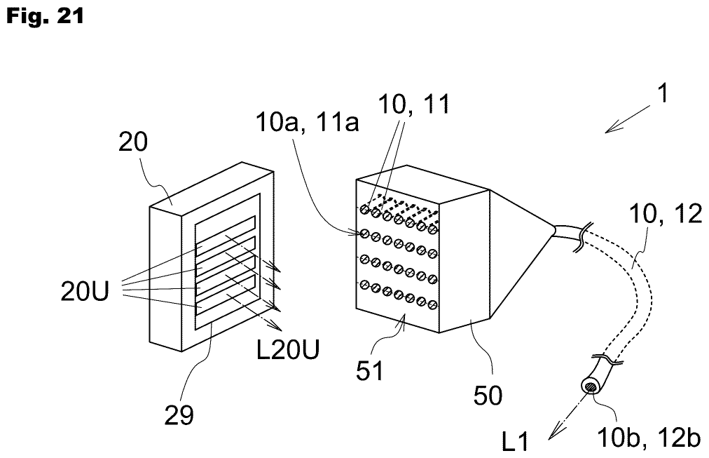

Further, as shown in FIG. 20, the light source unit 20 may include an ultraviolet light source 20U, a visible light source 20W, and an infrared light source 20I. Also in this case, as in the case shown in FIG. 14, the first light guide member 11 may have a plurality of branches at its incidence-side end so that lights from the respective light sources enter these branches, respectively. In this configuration, the visible light source 20W and the infrared light source 20I correspond to the “second light source”.
<3> FIG. 21 is a diagram schematically showing the configuration of another embodiment of the inactivation device 1. The light source unit 20 included in the inactivation device 1 includes a plurality of ultraviolet light sources 20U and a window member 29 that transmits ultraviolet light L20U from the ultraviolet light sources 20U. The ultraviolet light sources 20U are constituted from lamps.

The inactivation device 1 includes a light guide unit 50 used together with the light source unit 20. The light guide unit 50 houses a plurality of first light guide members 11, and incidence-side ends 11a of the respective first light guide members 11 faces a light capture surface 51. The light guide unit 50 includes a second light guide member 12 into which a combination of lights that have propagated in the respective first light guide members 11 enters.

The plurality of first light guide members 11 are disposed along a direction parallel to the longitudinal direction of the ultraviolet light source 20U. When the inactivation device 1 is used, the light guide unit 50 and the light source unit 20 are disposed so that the light capture surface 51 and the window member 29 come into contact with each other. In the case shown in FIG. 21, the light source unit 20 includes a plurality of ultraviolet light sources 20U, and therefore the plurality of first light guide members 11 are disposed along the longitudinal direction of each of the ultraviolet light sources 20U when the light capture surface 51 of the light guide unit 50 and the window member 29 of the light source unit 20 come into contact with each other.

Ultraviolet lights L20U emitted from the plurality of ultraviolet light sources 20U propagate through the plurality of first light guide members 11 in the light guide unit 50 so that ultraviolet light L1 is emitted through the emission-side end 12b of the second light guide member 12, that is, the second end 10b of the light guide body 10. The inactivation device 1 shown in FIG. 21 also makes it possible to locally irradiate a narrower area or a recessed portion with ultraviolet light L1.
<4> The above embodiments have been described with reference to a case where the ultraviolet light source 20U included in the light source unit 20 is an excimer lamp, but the ultraviolet light source 20U may be a solid-state light source such as an LED or a laser diode element.
<5> The configurations of the respective embodiments described above may appropriately be combined.

### [Second Configuration Example]

As a second configuration example of the present invention, embodiments of a discharge lamp will be described with reference to the drawings as appropriate.

### First Embodiment

A first embodiment of a discharge lamp according to the present invention will be described with reference to the drawings.

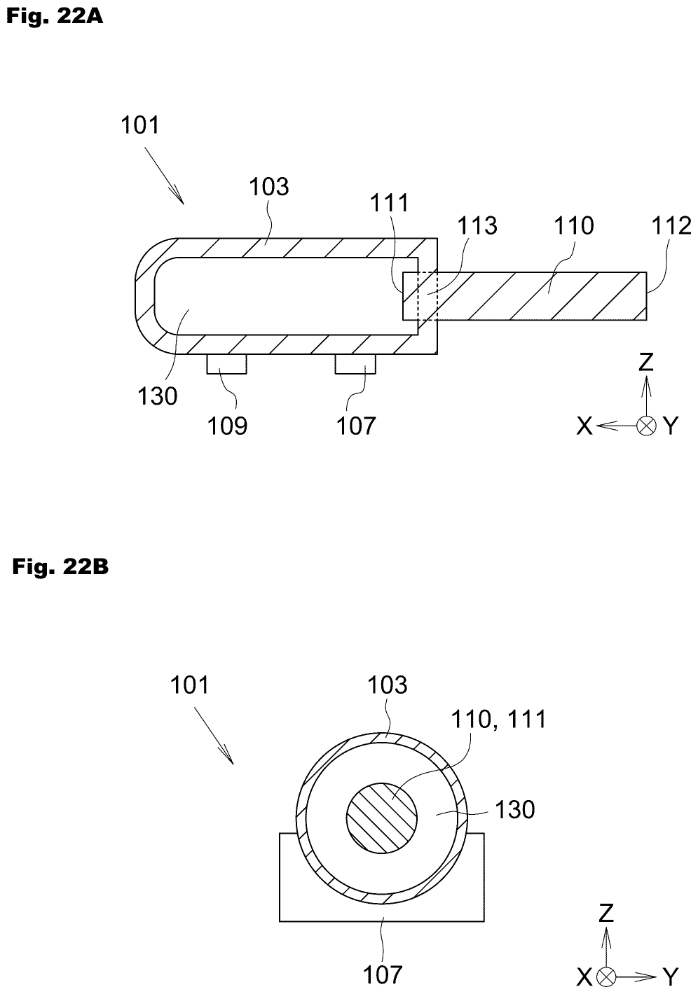

FIG. 22A is a cross-sectional view schematically showing a first embodiment of a discharge lamp 101. In the drawings described below, an X-Y-Z coordinate system including an X direction, a Y direction, and a Z direction orthogonal to each other is also indicated. When explained using this definition, FIG. 22A corresponds to a plan view when the cross-section of the discharge lamp 101 is viewed in the Y direction.

When it is necessary to make a distinction between positive and negative to express a direction herein, the direction is described with a positive or negative sign, such as “+X direction” or “−X direction”. When it is not necessary to make a distinction between positive and negative to express a direction, the direction is simply described as “X direction”. Namely, when the direction is simply described as “X direction” herein, both “+X direction” and “−X direction” are included. The same applies to the Y direction and the Z direction.

As shown in FIG. 22A, the discharge lamp 101 includes a light-emitting tube 103, a first electrode 107 and a second electrode 109 disposed on the tube wall of the light-emitting tube 103, and a light guide member 110 part of which is connected to the tube wall of the light-emitting tube 103. The light guide member 110 includes a first end 111 and a second end 112 on the side opposite to the first end 111, and is configured to extend from the first end 111 toward the second end 112.

The light-emitting tube 103 is formed of a dielectric material such as quartz glass and has an internal space 130 filled with a light-emitting gas containing, for example, KrCl. Typically, the light-emitting tube 103 is formed of synthesized quartz glass or fused quartz glass, and is preferably formed of synthesized quartz glass. In the present embodiment, the light-emitting tube 103 has an elongated shape whose longitudinal direction is parallel to the X direction.

FIG. 22B is a plan view when the discharge lamp 101 shown in FIG. 22A is viewed in the +X direction. As shown in FIG. 22A and FIG. 22B, in the first embodiment of the discharge lamp, the light-emitting tube 103 is a round tube having a circular shape when viewed in the X direction.

The discharge lamp 101 includes the light guide member 110 part of which is connected to the tube wall of the light-emitting tube 103. In FIG. 22A, a connection point 113 between the light guide member 110 and the light-emitting tube 103 is schematically shown by a broken line. As shown in FIG. 22A, in the first embodiment of the discharge lamp, the light guide member 110 is connected to the tube wall of the −X-side end of the light-emitting tube 103 and extends in the −X direction toward the second end 112 positioned outside the light-emitting tube 103 in the −X direction. The first end 111 of the light guide member 110 is exposed to the internal space 130 of the light-emitting tube 103.

From the viewpoint of ease of production, the light guide member 110 is preferably formed of a dielectric material such as quartz glass and is more preferably formed of the same material as the light-emitting tube 103.

On the tube wall of the light-emitting tube 103, the first electrode 107 and the second electrode 109 are disposed (see FIG. 22A). It should be noted that the second electrode 109 is disposed distant from the first electrode 107. FIG. 22A shows a case where the first electrode 107 and the second electrode 109 are disposed on the same surface of the tube wall (here, the −Z-side wall surface) of the light-emitting tube 103, and both the electrodes are distant from each other in the longitudinal direction (X direction) of the light-emitting tube 103.

As a main material of the first electrode 107 and the second electrode 109, a metallic material such as aluminum, copper, titanium, stainless steel, or brass can be used. The “main material” herein refers to a material that accounts for the highest proportion of materials constituting the electrode.

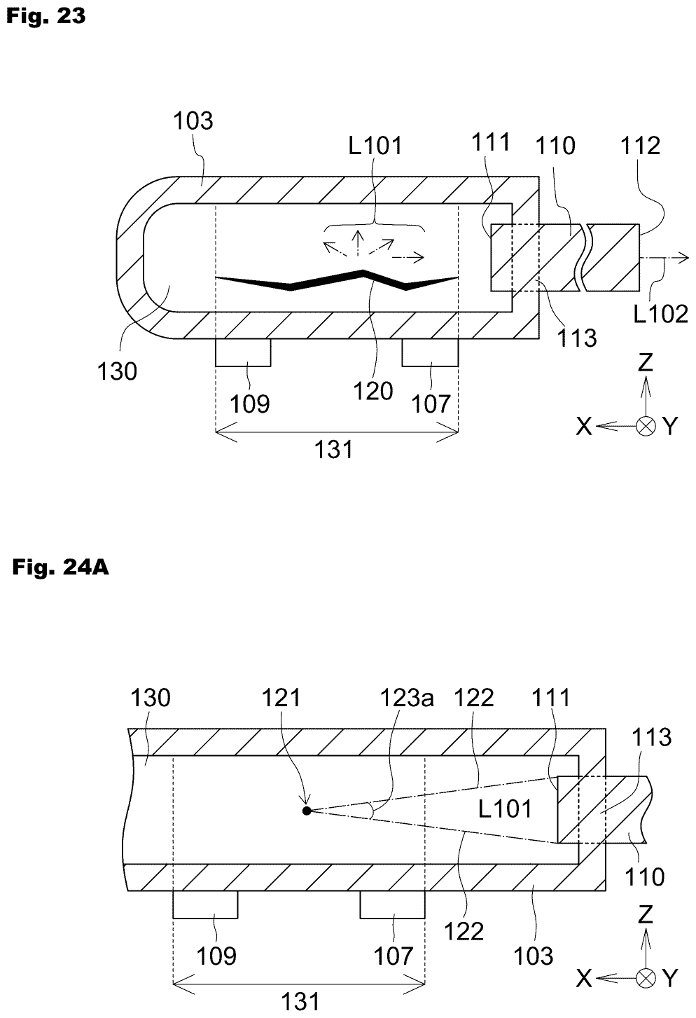

When high voltage with a high frequency is applied between the first electrode 107 and the second electrode 109, discharge plasma is generated in the internal space 130 of the light-emitting tube 103. This discharge plasma excites atoms or the like contained in the light-emitting gas filled in the internal space 130, and therefore light is radiated when the atoms or the like are de-excited from the excited state to the ground state. FIG. 23 is a conceptual diagram schematically showing discharge plasma 120 generated when high voltage with a high frequency is applied between the first electrode 107 and the second electrode 109.

As shown in FIG. 23, discharge plasma 120 is generated between an area facing the first electrode 107 and an area facing the second electrode 109 in the internal space 130. In FIG. 23, a space sandwiched between the area facing the first electrode 107 and the area facing the second electrode 109 in the internal space 130 of the light-emitting tube 103 is schematically shown and indicated by reference sign 131. The space indicated by reference sign 131 is hereinafter referred to as “effective discharge space”.

In FIG. 23, the traveling directions of light L101 radiated when atoms or the like excited by discharge plasma 120 are returned to the ground state are shown by a dash-dot line. As shown in FIG. 23, the light L101 travels in various directions. Part of the light L101 that travels toward a point where the first end 111 of the light guide member 110 is located is directly guided to the first end 111 and then enters the light guide member 110. The light L101 that has entered the light guide member 110 is extracted outside through the second end 112 as light L102.

The emission wavelength of light L101 depends on the energy levels of the excited state and the ground state of atoms or the like contained in the light-emitting gas. For example, when the light-emitting gas contains KrCl, ultraviolet light having a peak wavelength of around 222 nm can be obtained.

The present inventors have paid attention to the positional relationship between the first end 111 of the light guide member 110 exposed to the internal space 130 of the light-emitting tube 103 and the first electrode 107. From the viewpoint of extracting a larger amount of light L101 from the light-emitting tube 103, the first end 111 of the light guide member 110 is preferably brought close to the effective discharge space 131 in which discharge plasma 120 is generated so that light L101 is radiated.

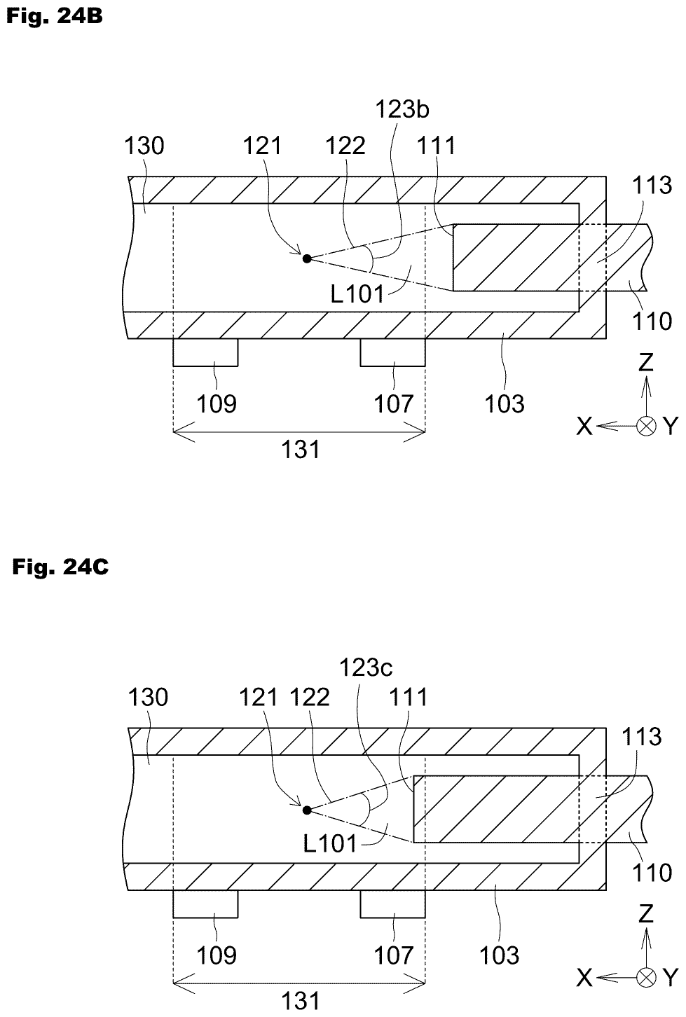

With reference to FIG. 24A and FIG. 24B, the behavior of light when the first end 111 of the light guide member 110 is brought close to the effective discharge space 131 will be described. FIG. 24A is a conceptual diagram showing the angular range of light L101 that can directly be captured by the first end 111 when light L101 is generated in the effective discharge space 131. FIG. 24B is a conceptual diagram showing the behavior of light L101 when the first end 111 is brought closer to the effective discharge space 131 as compared to the mode shown in FIG. 24A. That is, FIG. 24B shows a case where the first end 111 of the light guide member 110 is displaced in the +X direction from the position shown in FIG. 24A.

In FIG. 24A and FIG. 24B, a virtual point 121 from which light L101 is radiated is shown at the same position in the effective discharge space 131, and the range of an angle between virtual lines 122 connecting the virtual point 121 and the first end 111 (hereinafter referred to as “capturable angle” for convenience) is shown. That is, in FIG. 24A, light L101 radiated from the virtual point 121, and the light L101 traveling at an angle within the range of a capturable angle 123a is directly guided to the first end 111. It should be noted that in FIG. 24A and FIG. 24B, the virtual lines 122 are shown by a dash-dot line.

Referring to FIG. 24B, it can be understood that a capturable angle 123b shown in FIG. 24B is larger than the capturable angle 123a shown in FIG. 24A. That is, a larger amount of light L101 can be captured when the first end 111 is closer to the effective discharge space 131.

It should be noted that FIG. 24A and FIG. 24B show cases where the virtual point 121 is set around the center of the effective discharge space 131. However, the position where the virtual point 121 is set is not limited, and the above discussion can be applied to all the positions in the effective discharge space 131. Just in case, it should be added that in FIG. 24A and FIG. 24B, for ease of comprehension, the dimensions, etc. of the light-emitting tube 103 and the light guide member 110 are exaggeratedly shown.

FIG. 24C is a conceptual diagram showing a case where the first end 111 is further displaced in the +X direction as compared to the mode shown in FIG. 24B to dispose the light guide member 110 in such a manner that the first end 111 overlaps the effective discharge space 131. More specifically, the light guide member 110 is disposed in such a manner that the first end 111 and the first electrode 107 overlap each other in the Z direction corresponding to the normal direction of the tube wall (or the tangent plane of the tube wall) on which the first electrode 107 is disposed.

As in the case of the above discussion, when the first end 111 of the light guide member 110 is disposed to overlap the effective discharge space 131, a capturable angle 123c shown in FIG. 24C is larger than the capturable angle 123b shown in FIG. 24B. Therefore, the amount of light L101 that can be taken into the light guide member 110 in the mode shown in FIG. 24C is larger than that in the mode shown in FIG. 24B.

### [Verification]

The present inventors performed the following verification to investigate the effect of the positional relationship between the effective discharge space 131 and the first end 111 of the light guide member 110 on the irradiance of light L102 radiated from the second end 112.

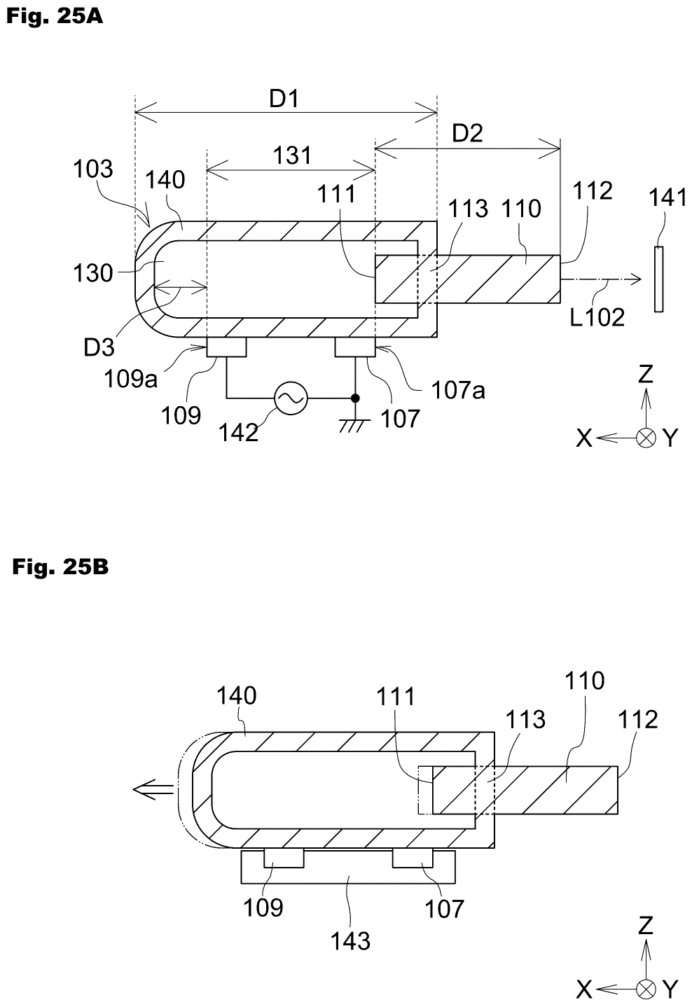

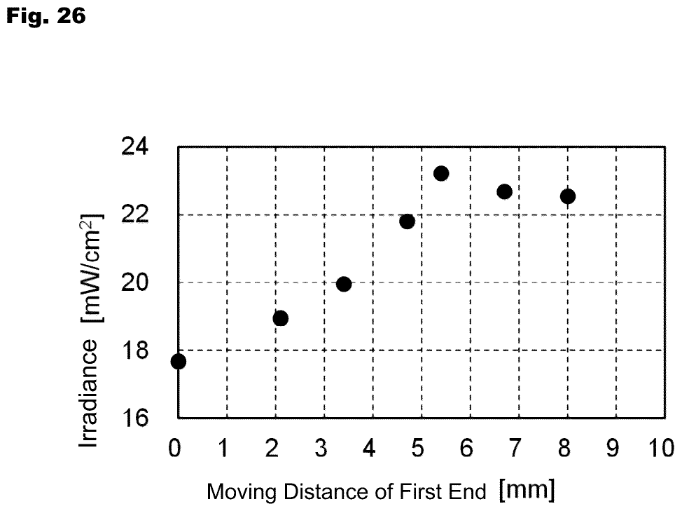

FIG. 25A is a conceptual diagram of an experimental system used for this verification. FIG. 25B is a conceptual diagram schematically showing an operation performed on a light-emitting tube 140 that will be described later. It should be noted that for the convenience of illustration, a stage 143 that will be described later is not shown in FIG. 25A. Further, in FIG. 25B, an alternating source 142 that will be described later is not shown.

First, as shown in FIG. 25A, a light-emitting tube 140 was prepared which was connected to a light guide member 110 so that a first end 111 was exposed to an internal space 130. Further, as a first electrode 107 and a second electrode 109, a pair of electrodes previously disposed on a stage 143 was used. The light-emitting tube 140 is disposed on the stage 143 to bring both the electrodes (107, 109) into contact with the −Z-side tube wall of the light-emitting tube 140 (see FIG. 25B).

In this verification, as shown in FIG. 25B, the first end 111 was moved relative to the first electrode 107 by moving the light-emitting tube 140 connected to the light guide member 110 in the +X direction relative to the stage 143 on which both the electrodes (107, 109) were disposed. In FIG. 25B, the positions of the light-emitting tube 140 and the first end 111 moved in the +X direction are shown by a dash-dot-dot line.

Since both the first electrode 107 and the second electrode 109 are fixed to the stage 143, when the light-emitting tube 140 is moved in the +X direction, a length D3 (see FIG. 25A) of the internal space 130 on the +X side from a +X-side end 109a of the second electrode 109 in the X direction increases. Therefore, there is a problem that when the positional relationship between the effective discharge space 131 and the first end 111 is changed by moving the light-emitting tube 140 in the +X direction, the length D3 is also changed. In light of such a problem, in order to strictly perform the verification by changing the positional relationship between the effective discharge space 131 and the first end 111, it is ideal that a plurality of light-emitting tubes different in the exposure distance of the first end 111 in the internal space 130 (see also FIG. 24A to FIG. 24C) are prepared.

As a supplement to this point, as described above, since discharge plasma 120 is generated in the effective discharge space 131 that is an area sandwiched between a space facing the first electrode 107 and a space facing the second electrode 109 in the internal space 130, a difference in the length D3 is considered not to have a major effect on the irradiance of light L102. That is, light-emitting tubes different in the exposure distance of the first end 111 in the internal space 130 can be simulated by moving the light-emitting tube 140 relative to both the electrodes (107, 109). Therefore, from the viewpoint of reducing the time and cost of the verification, this verification method was employed.

In this verification, the light-emitting tube 140 and the light guide member 110 were formed of synthesized quartz glass, and the internal space 130 was filled with a light-emitting gas containing KrCl at a pressure of 19 kPa. That is, in this experiment, an excimer lamp was used which emitted, through the second end 112, light L102 having a peak wavelength of around 222 nm.

The inner diameter of the light-emitting tube 140 as a round tube was 4.5 mm, and the outer diameter of the light guide member 110 having an almost cylindrical shape was 4 mm. An X-direction dimension D1 of the light-emitting tube 140 was 65 mm, and an X-direction dimension D2 of the light guide member 110 was 30 mm. Therefore, the dimensional ratio of the outer diameter of the light guide member 110 to the inner diameter of the light-emitting tube 140 is 0.9. It should be noted that typically, the inner diameter of the light-emitting tube 140 corresponds to the dimension of the internal space 130, and the outer diameter of the light guide member 110 corresponds to the dimension of the first end 111.

The first electrode 107 and the second electrode 109 used aluminum as a main material, and the X-direction dimension of each of the electrodes was 15 mm. The distance between both the electrodes (107, 109) in the X direction was 6 mm. That is, the X-direction length of the effective discharge space 131 was 36 mm.

In FIG. 25A, the first electrode 107 is connected to the ground side of the alternating source 142 with a high frequency of about 1 kHz to 5 MHz. That is, FIG. 25A corresponds to a case where the light guide member 110 is disposed on the side close to the first electrode 107 configured to be lower in electric potential than the second electrode 109 in terms of absolute value. On the other hand, as will be described later, another verification was also performed in which the low electric potential side and the high electric potential side of the alternating source 142 were reversed.

It should be noted that the irradiance of light L102 was measured using an irradiance meter 141 including a UV integrating photometer (UIT-250) manufactured by Ushio Inc. and a separate-type photodetector (VUV-S172, manufactured by Ushio Inc.) that had been calibrated using light with a wavelength of 222 nm. When the irradiance of light L102 was measured, the distance between the irradiance meter 141 and the second end 112 (in the X direction) was kept constant.

FIG. 26 is a graph obtained by plotting the irradiance of light L102 emitted through the second end 112, in which the vertical axis represents the irradiance of light L102 and the horizontal axis represents the moving distance of the first end 111 in the +X direction from an initial position (0 mm) where a −X direction-side end 107a of the first electrode 107 and the first end 111 of the light guide member 110 are at the same position in the X direction (see FIG. 25A). It should be noted that as described above with reference to FIG. 23, discharge plasma 120 is generated between an area facing the first electrode 107 and an area facing the second electrode 109 (i.e., in the effective discharge space 131) in the internal space 130. That is, an increase in an area where the light guide member 110 and the first electrode 107 overlap each other in the Z direction means a reduction in the effective discharge space 131 inside the light-emitting tube 140.

From the above hypothesis, it is considered that when the first end 111 of the light guide member 110 is moved relative to the first electrode 107 in the +X direction from the initial position corresponding to 0 mm in the graph, the amount of light emission reduces as the moving distance increases so that the irradiance reduces. However, as shown in FIG. 26, the actual result was that the irradiance of light L102 emitted through the second end 112 increased as the first end 111 of the light guide member 110 was moved relative to the first electrode 107 from the initial position in the +X direction. This result indicates that a larger amount of light can be extracted by disposing the first end 111 so as to overlap the effective discharge space 131.

When the irradiance of light L102 was continued to be measured while the moving distance was increased, as shown in FIG. 26, the irradiance of light L102 was maximized when the moving distance was 5.4 mm, and was then only slightly reduced when the moving distance was increased to 6.7 mm and then 8.0 mm.

Although the result when the moving distance was just 5 mm or 6 mm is not shown in FIG. 26, from the fact that the irradiance tends to increase when the moving distance increases from 0 mm to 5.4 mm but tends to only slightly reduce when the moving distance increases to 6.7 mm and then 8.0 mm, it is understood that a moving distance of 5 mm to 6 mm is effective at greatly increasing the irradiance of light L102. As described above, the X-direction dimension of the first electrode 107 is 15 mm. Therefore, the result shown in FIG. 26 indicates that a large amount of light L2 can be extracted from the light-emitting tube 103 when the first end 111 is disposed so that the ratio of the X-direction length of an area where the light guide member 110 and the first electrode 107 overlap each other in the Z direction to the X-direction dimension of the first electrode 107 is 0.33 to 0.4.

It should be noted that in FIG. 26, the reason why the irradiance of light L102 tends to only slightly reduce when the moving distance is increased to 6.7 mm and then 8.0 mm can be considered to be that, as described above, the effective discharge space 131 becomes narrow due to an increase in an area where the light guide member 110 and the first electrode 107 overlap each other in the Z direction. That is, it is presumed that an effect obtained by bringing the first end 111 of the light guide member 110 close to discharge plasma 120 (see FIG. 23) was relatively higher than an effect caused by narrowing the effective discharge space 131 until the first end 111 of the light guide member 110 reached a position 5 mm to 6 mm away from the initial position so that the irradiance of light L102 extracted was increased. On the other hand, it is presumed that when the first end 111 of the light guide member 110 was brought closer to the second electrode 109 beyond the above position, the effect caused by narrowing the effective discharge space 131 was increased so that the irradiance of light L102 started to reduce as the moving distance was increased. It can be understood that when the first end 111 of the light guide member 110 is brought much closer to the second electrode 109, the effective discharge space 131 is further narrowed, and therefore the amount of light emission itself is reduced so that the irradiance of light L102 extracted is reduced. Although not shown in FIG. 26, as a result, it has been confirmed that when the first end 111 of the light guide member 110 is brought very much closer to the second electrode 109, the irradiance of light L102 was further reduced than that when the moving distance was 8 mm.

At the same time, another verification was performed in which the ground side of the alternating source 142 was connected to the second electrode 109 to reverse the high electric potential side and the low electric potential side. That is, verification was performed for a case where the light guide member 110 was disposed on the side close to the first electrode 107 higher in electric potential than the second electrode 109 in terms of absolute value. As a result, the irradiance of light L102 was low as a whole. For example, in the case shown in FIG. 26, the irradiance when the moving distance of the first end 111 was 5.4 mm was 23 mW/cm², whereas, in such another verification, the irradiance of light L102 at the same position was lower than 20 mW/cm².

This confirmed that a larger amount of light L102 could be extracted from the light-emitting tube 140 when the light guide member 110 was disposed on the side close to the electrode lower in electric potential in terms of absolute value than when the light guide member 110 was disposed on the side close to the electrode higher in electric potential in terms of absolute value.

The reason for this is not clear, but for example, one of the presumable reasons is that large amounts of electrons contributing to the generation of discharge plasma 120 and ions derived from atoms or the like contained in light-emitting gas are present in the vicinity of the electrode higher in electric potential, and therefore discharge plasma 120 is more likely to be generated in the vicinity of the electrode as compared to the electrode lower in electric potential. That is, referring to FIG. 25A, in the vicinity of the second electrode 109 higher in electric potential, discharge plasma 120 is likely to be formed in the vicinity of the −Z-side tube wall of the light-emitting tube 140. On the other hand, in the vicinity of the first electrode 107 lower in electric potential, discharge plasma 120 is more likely to be formed around the center of the light-emitting tube 140 in the Z direction as compared to the electrode higher in electric potential. Accordingly, the present inventors have surmised that when the light guide member 110 is arranged closer to the first electrode 107 with the low potential side (experimental system in FIG. 25A), the amount of light captured at the first end 111 increases and a higher irradiance is achieved than when the light guide member 110 is arranged closer to the first electrode 107 with the high potential side (not shown).

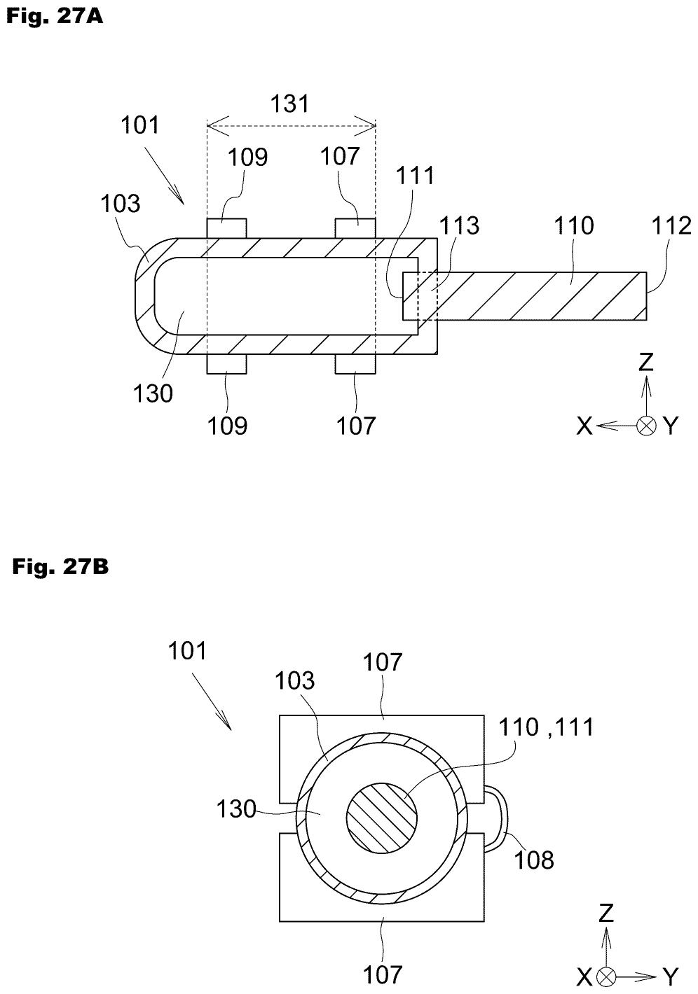

Hereinbelow, modifications of the present embodiment will be described.
<1> The present embodiment has been described above with reference to a case where the first electrode 107 and the second electrode 109 are disposed on the −Z direction-side wall surface of the light-emitting tube 103 (see FIG. 22A). However, as shown in, for example, FIG. 27A and FIG. 27B, the first electrode 107 and the second electrode 109 may be disposed not only on the −Z direction-side wall surface but also on the +Z direction-side wall surface of the light-emitting tube 103. FIG. 27A is a cross-sectional view schematically showing, in the same manner as FIG. 22A, the structure of a discharge lamp as such another configuration example. FIG. 27B is a plan view when the discharge lamp shown in FIG. 27A is viewed in the +X direction. As shown in FIG. 27A, since the second electrode 109 is positioned on the +X side from the first electrode 107, only the first electrode 107 is shown in FIG. 27B. Actually, the second electrode 109 is disposed in the same manner as the first electrode 107 at a position on the +X side from the position shown in FIG. 27B.

As described above, discharge plasma 120 (see FIG. 23) is generated in the effective discharge space 131 sandwiched between an area facing the first electrode 107 and an area facing the second electrode 109 in the internal space 130. Therefore, as shown in FIG. 27A and FIG. 27B, when the first electrodes 107 are disposed so as to sandwich the light-emitting tube 103 in, for example, the Z direction, a space where discharge plasma 120 is generated in the effective discharge space 131 can be increased. It should be noted that when being discontinuously disposed as shown in FIG. 27B, the first electrodes 107 are electrically connected by, for example, an electrically conductive member 108 so that all the first electrodes 107 are at the same electric potential.

Further, as shown in FIG. 27B, when the first electrodes 107 are disposed so as to sandwich the light-emitting tube 103 in the Z direction, there is also a merit that the effect of tolerance of the light-emitting tube 103 is relatively reduced and the first electrodes 107 can easily be brought into close contact with the light-emitting tube 103.

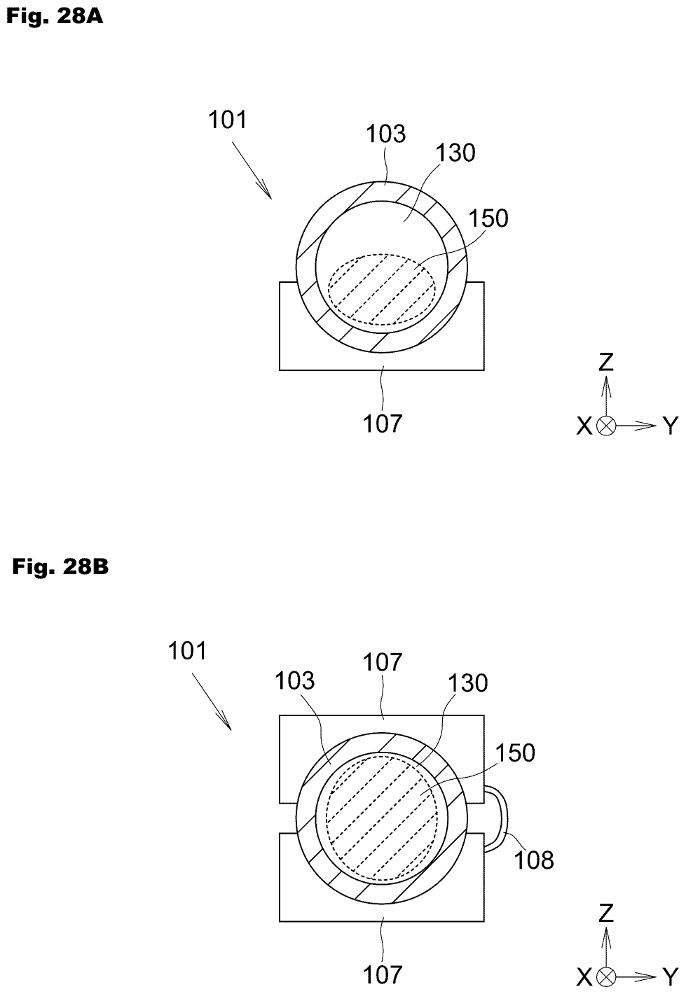

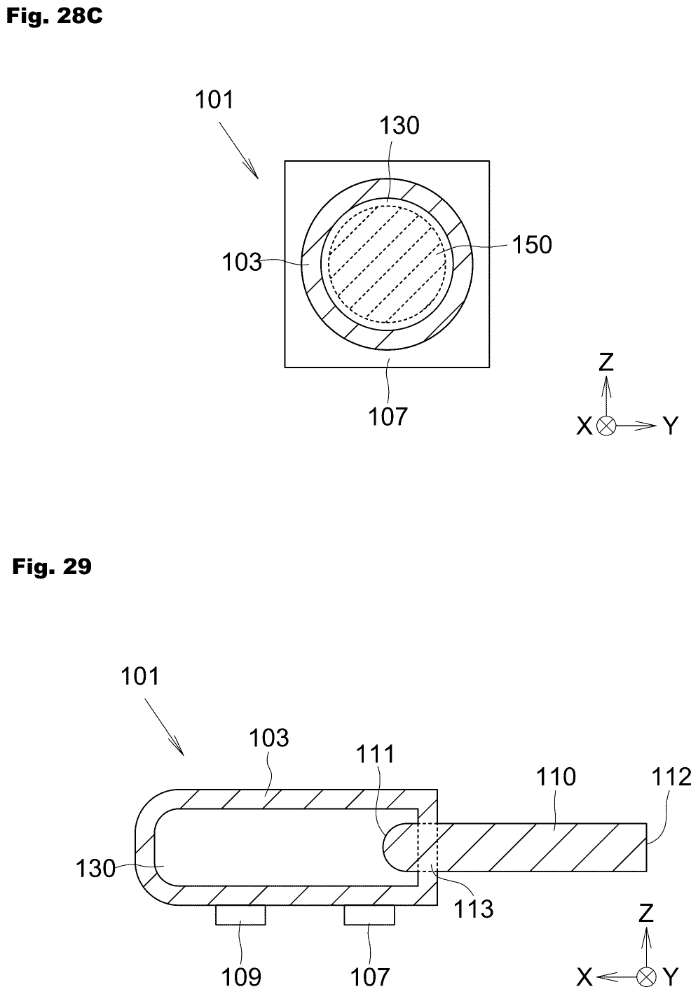

Referring to FIG. 28A to FIG. 28C, the merit of using the structure of such another configuration example will be described. FIG. 28A is a conceptual diagram schematically showing, for comparison, an area where discharge plasma 120 is mainly generated in the effective discharge space 131, under the structure of the discharge lamp 101 shown in FIG. 22A, i.e., under the structure where the first electrode 107 is disposed on the −Z direction-side wall surface of the light-emitting tube 103 (see also FIG. 22B). FIG. 28A is a conceptual diagram when the internal space 130 of the light-emitting tube 103 is viewed in the X direction, in which a space where discharge plasma 120 is mainly generated (hereinafter referred to as “virtual discharge area 150” for convenience) is hatched by broken lines. On the other hand, FIG. 28B corresponds to a diagram showing an area where discharge plasma 120 is mainly generated in the effective discharge space 131, under the structure of the discharge lamp 101 of another configuration example shown in FIG. 27A, i.e., under the structure where the first electrodes 107 are disposed not only on the −Z direction-side wall surface but also on the +Z direction-side wall surface of the light-emitting tube 103 (see also FIG. 27B).

In FIG. 28A, since the first electrode 107 is disposed only on the −Z direction-side wall surface of the light-emitting tube 103, discharge plasma 120 is mainly generated at a local position on the −Z direction side. On the other hand, in FIG. 28B, since the first electrodes 107 are disposed on the −Z direction-side wall surface and the +Z direction-side wall surface of the light-emitting tube 103, discharge plasma 120 is widely generated in the internal space 130 in the entire Z direction without being unevenly distributed.

As described above, when the first electrodes 107 are disposed in areas facing each other across the internal space 130 of the light-emitting tube 103 (here, in the Z direction), discharge plasma 120 is generated entirely in the effective discharge space 131, and therefore a larger amount of light can be taken in from the light guide member 110. This point conforms also to the presumption obtained by the above-described verification, that is, the presumption that it is preferred that discharge plasma 120 is formed around the center of the light-emitting tube 103 in the Z direction.

From the same viewpoint, as shown in FIG. 28C, the first electrode 107 may be disposed so as to cover the entire circumference of the light-emitting tube 103. From the viewpoint of generating discharge plasma 120 entirely in the effective discharge space 131, it is more preferred that the first electrode 107 covers the entire circumference of the light-emitting tube 103.

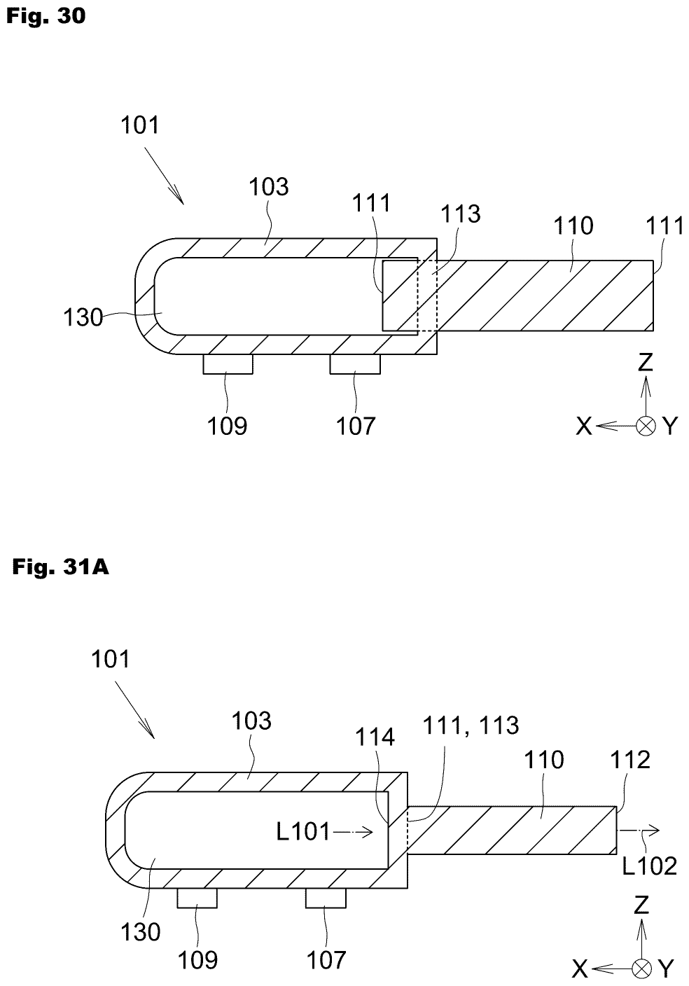

It should be noted that the above discussion has been made for the first electrode 107, but the same discussion can be applied to the second electrode 109. That is, from the viewpoint of effectively generating discharge plasma 120 in the effective discharge space 131, as shown in FIG. 27A, it is preferred that both the first electrode 107 and the second electrode 109 are disposed not only on the −Z direction-side wall surface but also on the +Z direction-side wall surface of the light-emitting tube 103.
<2> The present embodiment has been described above with reference to a case where the end surface of the first end 111 of the light guide member 110 has a planar shape, but as shown in FIG. 29, the end surface of the first end 111 may be constituted from a curved surface that protrudes toward the internal space 130. An example of such a curved surface is part of a spherical surface or an oval spherical surface.
<3> From the viewpoint of extracting a larger amount of light L101 from the light-emitting tube 103, as shown in, for example, FIG. 30, it is preferred that the Z-direction dimension of the internal space 130 in the light-emitting tube 103 and the Z-direction dimension of the first end 111 of the light guide member 110 are almost the same. Specifically, an error in dimension between them is preferably in the range of 20% or less.

### Second Embodiment

A second embodiment of the discharge lamp according to the present invention will be described by mainly focusing on different points from the first embodiment. FIG. 31A shows the discharge lamp 101 of the present embodiment in the same manner as FIG. 22A.

As described above with reference to FIG. 22A, the first embodiment is a case where the first end 111 of the light guide member 110 is exposed to the internal space 130 of the light-emitting tube 103. However, as shown in FIG. 31A, the first end 111 of the light guide member 110 may be connected to the tube wall of the light-emitting tube 103.

Specifically, in FIG. 31A, light L101 generated in the light-emitting tube 103 is guided to the light guide member 110 through a region, which faces the first end 111 of the light guide member 110 (hereinafter referred to as “incidence region 114” for convenience), in the inner wall of the light-emitting tube 103. Also in this case, light L101 can efficiently be extracted through the incidence region 114 because the light guide member 110 is connected to the tube wall of the light-emitting tube 103. FIG. 31B is a plan view when the incidence region 114 is viewed from the internal space 130 of the light-emitting tube 103 in the −X direction, in which a region corresponding to the incidence region 114 is hatched by broken lines.

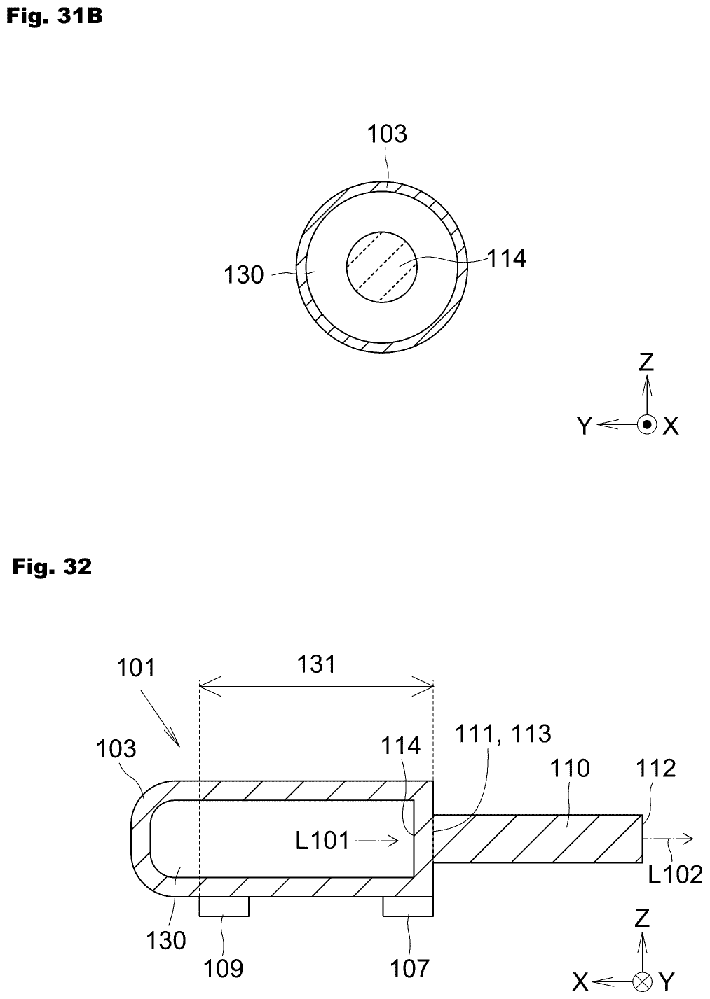

In the present embodiment, the effective discharge space 131 is preferably brought close to the incidence region 114 (see FIG. 32). More specifically, the discharge lamp 101 is preferably configured in such a manner that the inner wall of the light-emitting tube 103 at a position facing the first end 111 of the light guide member 110 (the incidence region 114) and the first electrode 107 overlap each other in the Z direction. This makes it possible to more efficiently extract light from the light-emitting tube 103 because the incidence region 114 is disposed so as to overlap the effective discharge space 131.

### OTHER EMBODIMENTS

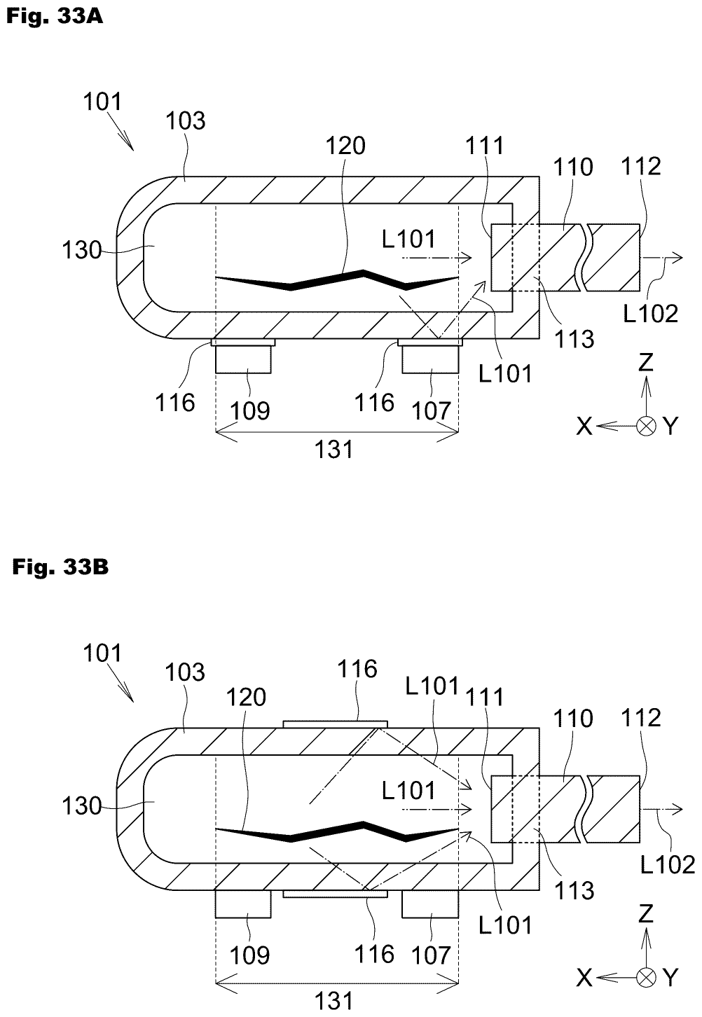

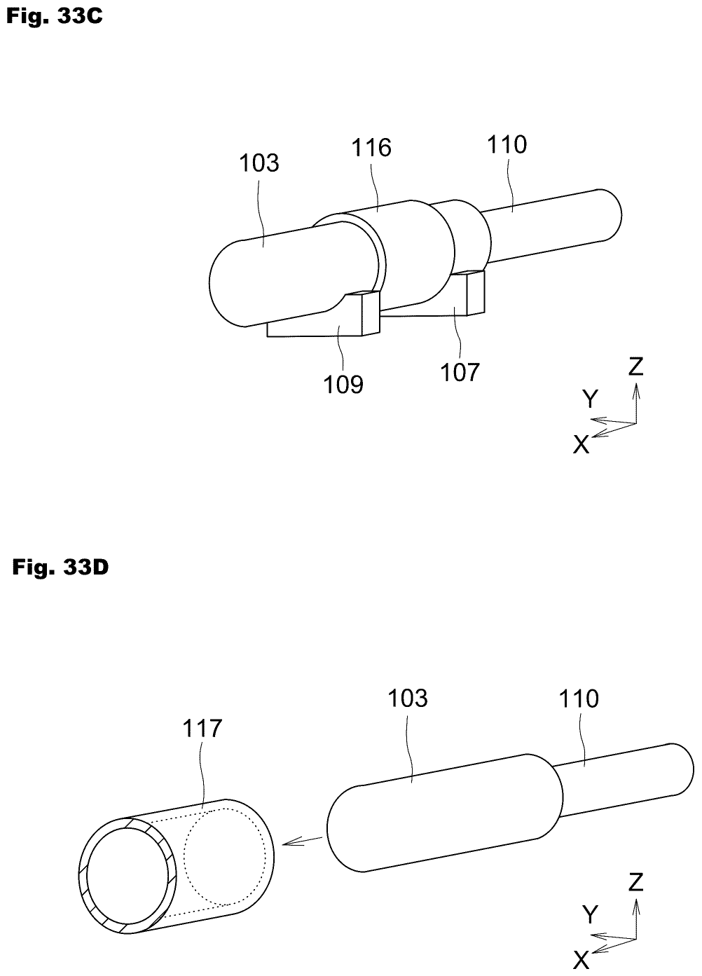

Hereinbelow, other embodiments will be described. It should be noted that structures described below with reference to other embodiments can be combined with the respective embodiments described above as appropriate.
<1> As shown in FIG. 33A, a reflective layer 116 may be provided at each of the interfaces between the first electrode 107 and the tube wall of the light-emitting tube 103 and the interface between the second electrode 109 and the tube wall of the light-emitting tube 103. Here, the first electrode 107 and the second electrode 109 have a certain reflectance to light L101, but there is a case where the reflectance to light L101 of the surface of each of the electrodes (107, 109) is reduced depending on the wavelength of light L101 or the material and processing accuracy of each of the electrodes (107, 109). For example, when the electrodes have microscopic surface irregularities, there is a possibility that light entering the surface of each of the electrodes (107, 109) diffuses and reflects so that the proportion of light that returns to the inside of the light-emitting tube 103 as optical feedback is reduced. On the other hand, as shown in FIG. 33A, when the reflective layer 116 is provided at each of the interfaces between the first electrode 107 and the tube wall of the light-emitting tube 103 and the interface between the second electrode 109 and the tube wall of the light-emitting tube 103, much of light L101 generated in the light-emitting tube 103 and traveling toward the first electrode 107 or the second electrode 109 can return to the inside (internal space 130) of the light-emitting tube 103 and appropriately enter the light guide member 110.

The reflective layer 116 may be a sheet member formed of a metal such as aluminum. The reflective layer according to the above configuration can be achieved through a simple production process by sandwiching the above-described sheet member between the tube wall of the light-emitting tube and the electrode or by forming a reflective film on the surface of the electrode.

It should be noted that the reflective layer 16 may be provided by forming a ceramic coating film containing silica particles or the like or a reflective film such as a dielectric multi-layer film formed by laminating dielectric material layers different in refractive index on at least one selected from among the tube wall of the light-emitting tube 103, the surface of the first electrode 107, and the surface of the second electrode 109.

That is, the reflective layer 116 can appropriately be designed according to the type of material used for each of the electrodes (107, 109) or the wavelength of light L101.

The present invention does not exclude a structure in which the reflective layer 116 is provided only at the interface between one of the electrodes and the tube wall of the light-emitting tube 103.
<2> As shown in FIG. 33B, the reflective layer 116 may be disposed between the first electrode 107 and the second electrode 109 in the X direction. FIG. 33C is a perspective view of the discharge lamp 101 shown in FIG. 33B. As shown in FIG. 33C, in this embodiment, the reflective layer 116 is formed so as to cover the entire circumference of the light-emitting tube 103.

As described above, discharge plasma 120 is generated in the effective discharge space 131 so that light L101 is radiated. Therefore, as shown in FIG. 33B, when the reflective layer 116 is provided between both the electrodes (107, 109) in the X direction, part of light L101 that travels in directions different from the direction of a point where the first end 111 is located, and typically passes through the light-emitting tube 103 can appropriately enter the light guide member 110.

Such a reflective layer 116 can be formed by, for example, winding a sheet member formed of aluminum or a sheet member formed of a fluorine-based resin material such as PTFE around the light-emitting tube 103. Alternatively, as shown in FIG. 33D, such a reflective layer 116 may be formed by, for example, inserting the light-emitting tube 103 into a tubular member 117 formed of a fluorine-based resin material such as PTFE. As described above, the reflective layer 116 can be achieved by a simple process by utilizing the above-described sheet member or tubular member. FIG. 33D is a perspective view conceptually showing a mode in which the light-emitting tube 103 connected to the light guide member 110 is inserted into the tubular member 17 constituting the reflective layer 116.

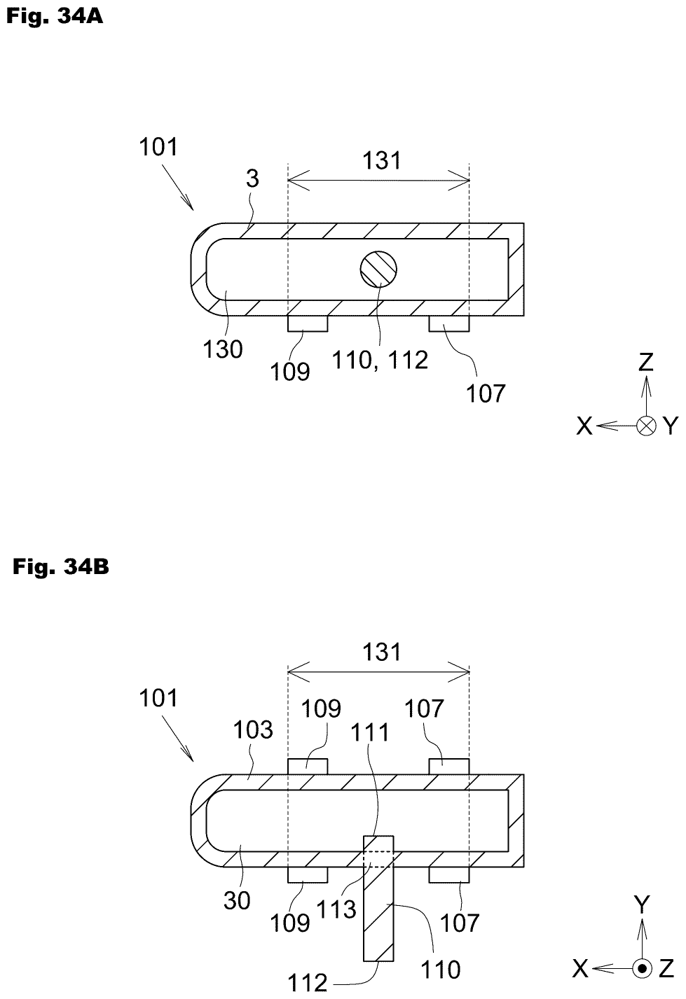

The above-described coating film or dielectric multi-layer film may be formed on the tube wall of the light-emitting tube 103. It should be noted that FIG. 33B and FIG. 33C show cases where the reflective layer 116 is formed on the outer wall of the light-emitting tube 103, but the reflective layer 116 may be formed on the inner wall of the light-emitting tube 103. For example, a coating film containing silica particles, PTFE particles, or the like may be formed on the inner wall of the light-emitting tube 103.
<3> The reflective layer 116 may be disposed in both a region where the first electrode 107 and the second electrode 109 are formed and a region between both the electrodes (107, 109). It should be noted that the reflective layer 116 may be disposed in a region other than these regions.
<4> The respective embodiments described above are cases where the light guide member 110 is connected to the light-emitting tube 103 at the longitudinal direction (X direction)-side end of the light-emitting tube 103. However, the present invention is not limited to such a structure. For example, as shown in FIG. 34A and FIG. 34B, the end of the light-emitting tube 103 to which the light guide member 110 is connected may be in a direction (here, the Y direction) different from the longitudinal direction of the light-emitting tube 103. It should be noted that FIG. 34B is a cross-sectional view when the discharge lamp 101 shown in FIG. 34A is viewed in the −Z direction.

Specifically, the first electrode 107 and the second electrode 109 are disposed distant from each other in the X direction on the same wall surface of the light-emitting tube 103, and the light guide member 110 is connected to the light-emitting tube 103 at a position between the first electrode 107 and the second electrode 109 in the X direction. In the case of such a configuration, the first end 111 of the light guide member 110 is disposed in the effective discharge space 131, and therefore light L101 can efficiently be taken into the light guide member 110. It should be noted that the same applies to a case where the first end 111 of the light guide member 110 is connected to the wall surface of the light-emitting tube 103 at a position between the first electrode 107 and the second electrode 109.

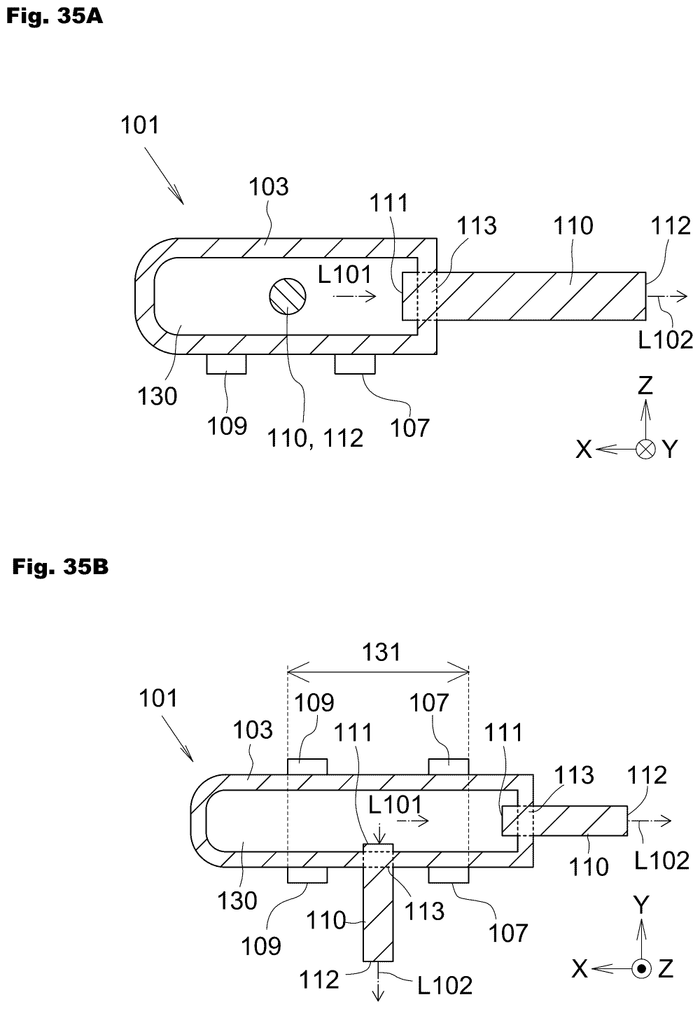

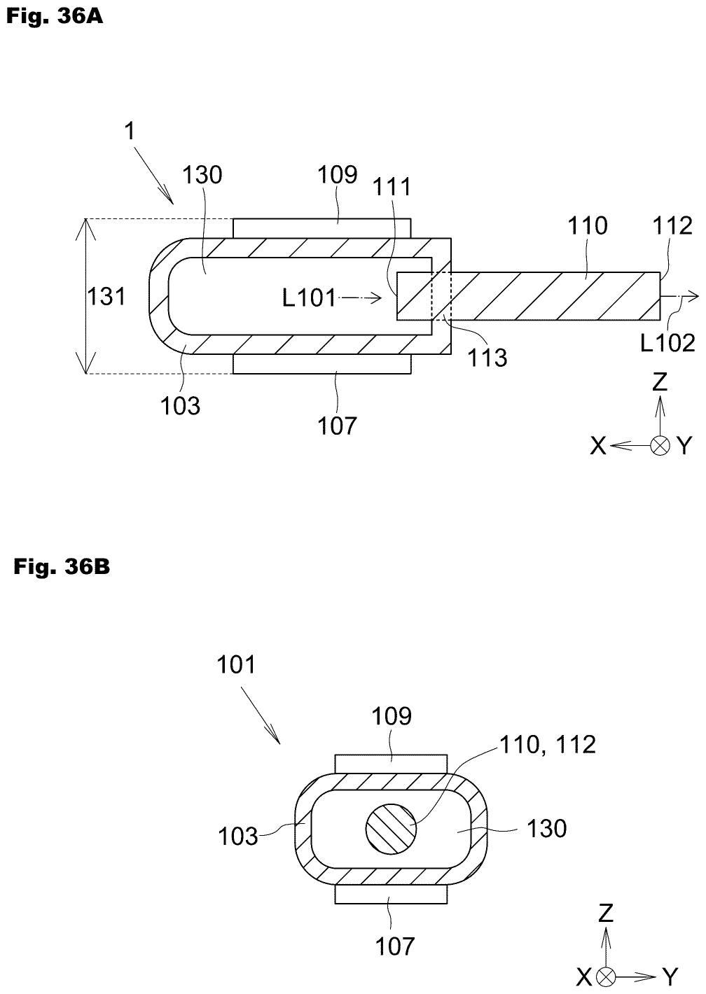

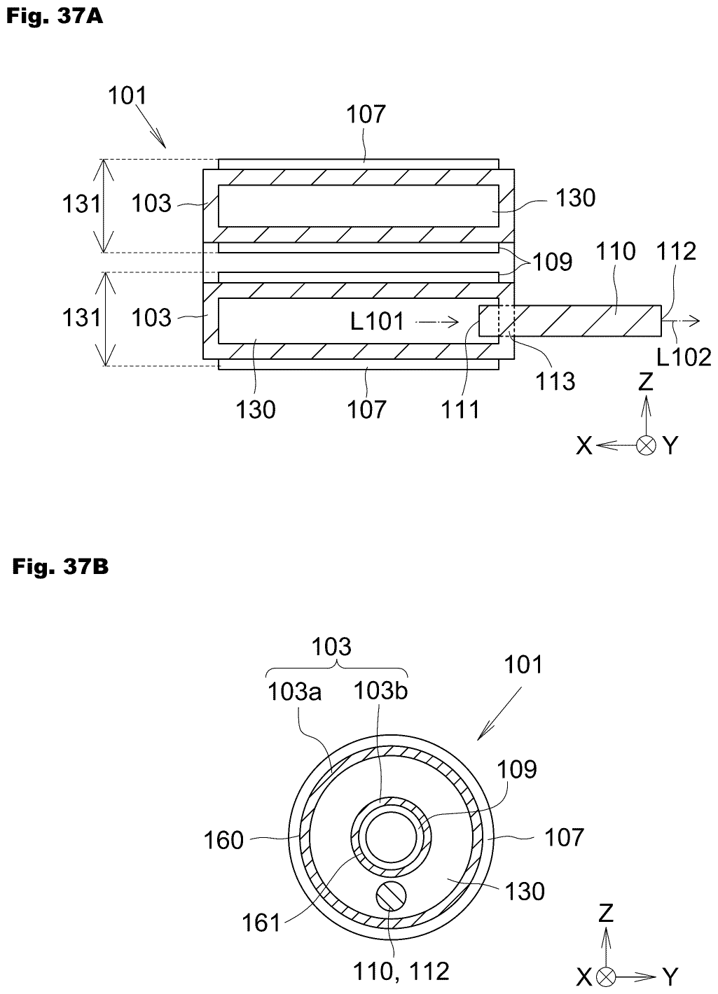

It should be noted that as shown in FIG. 35A and FIG. 35B, a plurality of light guide members 110 may be provided for the light-emitting tube 103. FIG. 35 shows a case where the light guide members 110 are connected to the tube wall of −X-side end of the light-emitting tube 103 and the tube wall of −Y-side end of the light-emitting tube 103, respectively. FIG. 35B is a plan view when the discharge lamp 101 shown in FIG. 35A is viewed in the −Z direction. As described above, when the light guide members 110 are connected in different directions, light L101 radiated from the internal space 130 can appropriately be taken into the light guide members 110.
<5> The embodiments described above are cases where the first electrode 107 and the second electrode 109 are disposed distant from each other in the X direction. Unlike such a structure, as shown in FIG. 36A and FIG. 36B, the first electrode 107 and the second electrode 109 may be disposed distant from each other in the Z direction. FIG. 36B is a plan view when the discharge lamp 101 shown in FIG. 36A is viewed in the X direction. As shown in FIG. 36B, the light-emitting tube 103 may be a flat tube. Also in this case, the effective discharge space 131 and the first end 111 of the light guide member 110 are preferably disposed to be close to each other, and more preferably, both of them overlap each other. The same discussion as described above can be applied to this point.
<6> As shown in FIG. 37A and FIG. 37B, the discharge lamp 101 may have a double-tube structure. FIG. 37A shows the discharge lamp 101 of this embodiment in the same manner as FIG. 22A. FIG. 37B is a plan view when the discharge lamp 101 shown in FIG. 37A is viewed in the X direction. As shown in FIG. 37B, the light-emitting tube 103 according to this embodiment has a ring shape when viewed in the X direction. This embodiment is a case where the light guide member 110 is connected to the tube wall of −X-side end of the light-emitting tube 103, and the first end 111 is exposed to the internal space 130 of the light-emitting tube 103.

As shown in FIG. 37B, the first electrode 107 is disposed on an outer wall surface 160 of the light-emitting tube 103 (in FIG. 37B, indicated by reference numeral 3a for convenience) along the circumferential direction of the light-emitting tube 103. Further, the second electrode 109 is disposed on an inner wall surface 161 of the light-emitting tube 103 (in FIG. 37B, indicated by reference numeral 3b for convenience) along the circumferential direction of the light-emitting tube 103. The same discussion about the positional relationship between the effective discharge space 131 and the first end 111 of the light guide member 110 as described above can be applied also to this case.
<7> The embodiments described above are cases where the light-emitting gas is KrCl, but the type of light-emitting gas is not limited in the present invention. As a typical example, the light-emitting gas may be at least one selected from the group consisting of KrCl, Ar₂, Kr₂, Xe₂, KrBr, XeCl, and XeBr.
<8> The configurations of the respective embodiments and modifications described above may be combined as appropriate.

### DESCRIPTION OF REFERENCE SIGNS

1 Inactivation apparatus
3 Housing body
5 End projecting part
7 Optical filter
10 Light guide body
10a First end of light guide body
10b Second end of light guide body
11 First light guide member
12 Second light guide member
13 Third light guide member
15 Flexible member
20 Light source unit
20I; Infrared light source
20U Ultraviolet light source
20W Visible light source
21 Light-emitting tube
23 Electrode
24 Electrode
25 Light-emitting space
27 Condenser lens
28 Condensing reflector
29 Window member
31 Power-supply unit
32 Control unit
40 Endoscope
41 Connector
42 Operating part
43 Insertion part
46 Treatment tool
47 Objective lens
48 Suction port
50 Light guide unit
51 Light capture surface
101 Discharge lamp
103 Light-emitting tube
107 First electrode
107a End of first electrode
108 Electrically conductive member
109 Second electrode
109a End of second electrode
110 Light guide member
111 First end of light guide member
112 Second end of light guide member
113 Connection point
114 Incidence region
116 Reflective layer
117 Tubular member
120 Discharge plasma
121 Virtual point
122 Virtual line
123a, 123b, 123c Capturable angle
130 Internal space
131 Effective discharge space
140 Light-emitting tube
141 Irradiance meter
142 Alternating source
143 Stage
150 Virtual discharge area
160 Outer wall surface
161 Inner wall surface
L1 Ultraviolet light
L20I Infrared light
L20U Ultraviolet light
L20W Visible light
L101, L102 Light

1. A bacteria or viruses inactivation device comprising:
a first light source that emits ultraviolet light having a principal wavelength band at least part of which is within a range of 200 nm or more and less than 240 nm;
a housing body that houses the first light source; and
a light guide body that has an elongated shape and guides the ultraviolet light emitted from the first light source in a longitudinal direction, part of the light guide body, including a first end that is an end on a side close to the light source, being positioned in the housing body,
wherein the light guide body is disposed in such a manner that a second end, which is an end on a side opposite to the first end, projects outside the housing body.

2. The bacteria or viruses inactivation device according to claim 1, wherein the light guide body includes an optical member that guides the ultraviolet light to the second end by internal total reflection.

3. The bacteria or viruses inactivation device according to claim 1, comprising an optical filter that is disposed at at least one position selected from among the first end, the second end, and an intermediate position between the first end and the second end of the light guide body to prevent travel of a wavelength component within a range of 240 nm or more and less than 280 nm that the ultraviolet light contains.

4. The bacteria or viruses inactivation device according to claim 1, wherein the second end of the light guide body is convex outward.

5. The bacteria or viruses inactivation device according to claim 1, comprising a flexible member that covers the second end of the light guide body and has permeability to the ultraviolet light.

6. The bacteria or viruses inactivation device according to claim 1, wherein the light guide body includes, at a region closer to the second end than the first end, a region whose outer diameter decreases toward the second end.

7. The bacteria or viruses inactivation device according to claim 1, wherein the light guide body is formed by connecting a plurality of light guide members in series to each other.

8. The bacteria or viruses inactivation device according to claim 7, wherein at least one of the plurality of light guide members constituting the light guide body, which is disposed at a position closest to the second end, includes an optical fiber or a light guide.

9. The bacteria or viruses inactivation device according to claim 1, comprising a light collection optical system that collects the ultraviolet light emitted from the first light source and guides it to the first end of the light guide body.

10. The bacteria or viruses inactivation device according to claim 1, wherein the first light source is a lamp filled with a gas formed of a material containing at least one of KrCl and KrBr.

11. The bacteria or viruses inactivation device according to claim 1, comprising a second light source that is housed in the housing body and has a principal wavelength band that is out of a range of 200 nm or more and less than 240 nm but is within at least one of a visible light range and an infrared range,
wherein the light guide body guides light emitted from the second light source to the second end at a timing the same as or different from the ultraviolet light emitted from the first light source.

12. A treatment device comprising the bacteria or viruses inactivation device according to claim 1, wherein a treatment site is irradiated with the ultraviolet light emitted through the second end of the light guide body.

13.-25. (canceled)

26. The bacteria or viruses inactivation device according to claim 1, wherein
the first light source is a lamp including a light-emitting tube formed of a dielectric material and having an inside filled with a light-emitting gas, and
the first end of the light guide body is connected to the light-emitting tube of the lamp.

27. The bacteria or viruses inactivation device according to claim 26, wherein at least a region, which is on a side close to the first end, in the light guide body is formed of the same material as the light-emitting tube of the lamp.
